(19) United States
(12) Patent Application Publication
(10) Pub. No.: US 2025/0120614 A1
(43) Pub. Date: Apr. 17, 2025

(54) INFORMATION PROCESSING APPARATUS, DISPLAY SYSTEM, DISPLAY CONTROL METHOD, AND NON-TRANSITORY COMPUTER READABLE MEDIUM STORING PROGRAM

(71) Applicants: NEC Corporation, Tokyo (JP); NATIONAL UNIVERSITY CORPORATION TOKYO MEDICAL AND DENTAL UNIVERSITY, Tokyo (JP)

(72) Inventors: Makoto YASUKAWA, Tokyo (JP); Yuji OHNO, Tokyo (JP); Yuki KOSAKA, Tokyo (JP); Hirofumi TAKASE, Tokyo (JP); Junpei KATOU, Tokyo (JP); Akimoto NIMURA, Tokyo (JP); Koji FUJITA, Tokyo (JP); Takuya IBARA, Tokyo (JP)

(73) Assignees: NEC Corporation, Tokyo (JP); NATIONAL UNIVERSITY CORPORATION TOKYO MEDICAL AND DENTAL UNIVERSITY, Tokyo (JP)

(21) Appl. No.: 18/685,262

(22) PCT Filed: Jul. 25, 2022
(86) PCT No.: PCT/JP2022/028571

§ 371 Date: Feb. 21, 2024

(30) Foreign Application Priority Data
Aug. 25, 2021 (JP) 2021-136990

(51) Int. Cl. A61B 5/113; G06F 3/14

(52) U.S. Cl. CPC A61B 5/113; G06F 3/14

(57) ABSTRACT

An acquisition unit acquires image data indicating at least a chest and an abdomen of a test subject. A detection unit detects the displacement of each of the chest and abdomen of the test subject by using the image data. A display control unit performs control so that the movement image of each of the chest and abdomen is displayed based on the detected displacement of the chest and abdomen. When the movement of the chest and the movement of the abdomen are synchronized with each other, the display control unit performs control so that the display form of the chest movement image and the display form of the abdomen movement image are the same as each other.

### TECHNICAL FIELD

The present invention relates to an information processing apparatus, a display system, a display control method, and a non-transitory computer readable medium storing a program.

### BACKGROUND ART

In order to improve and maintain the state of health, it is desirable to perform breathing with the correct breathing technique. In order to confirm whether or not the patient is breathing properly, chest (thorax) and abdominal (abdomen) movements are detected. In regard to this, Patent Literature 1 discloses that chest and abdominal movements are measured from images obtained by photographing the chest and abdomen of a breathing patient. Non-Patent Literature 1 discloses that natural breathing movements are measured in a non-contact manner by pattern light projection onto the trunk.

### CITATION LIST

### Patent Literature

Patent Literature 1: Japanese Unexamined Patent Application Publication No. 2008-154655

### Non Patent Literature

Non-Patent Literature 1: Hidetoshi Nakamura, “[Special issue] New developments in respiratory function imaging ‘Non-contact measurement of respiration by pattern light projection method’,” Respiratory Clinical, Vol. 2, No. 10, 2018

### SUMMARY OF INVENTION

### Technical Problem

For proper breathing training, it is important that the anteroposterior chest movements and the anteroposterior abdominal movements of a test subject synchronize with each other. In addition, it is desirable that the test subject can easily recognize whether or not he/she is breathing correctly. The technologies described above in the Patent Literature and Non-Patent Literature are intended for the physician or the like to diagnose the respiratory condition (i.e., breathing condition) of the patient. In other words, the technologies described above in the Patent Literature and Non-Patent Literature do not assume that the patient will understand his/her own respiratory condition. Therefore, it is difficult for the patient (test subject) to recognize whether or not he/she is breathing correctly even when he/she looks at the analysis screen disclosed in the above Patent Literature and Non-Patent Literature.

The present disclosure has been made to solve the above-described problem, and an object thereof is to provide an information processing apparatus, a display control method, and a program capable of enabling the test subject to easily recognize whether or not the test subject is breathing correctly.

### Solution to Problem

An information processing apparatus according to the present disclosure includes: acquisition means for acquiring image data indicating at least a chest and an abdomen of a test subject performing training of breathing; detection means for detecting displacement of each of the chest and abdomen of the test subject by using the image data; and display control means for performing control so that movement image indicating movement of each of the chest and abdomen in anteroposterior direction of the test subject is displayed based on the detected displacement of the chest and abdomen, wherein the display control means performs control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

Further, a display control method according to the present disclosure includes: acquiring image data indicating at least a chest and an abdomen of a test subject performing training of breathing; detecting displacement of each of the chest and abdomen of the test subject by using the image data; performing control so that movement image indicating movement of each of the chest and abdomen in anteroposterior direction of the test subject is displayed based on the detected displacement of the chest and abdomen; and performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

Further, a program according to the present disclosure causes a computer to perform: a step of acquiring image data indicating at least a chest and an abdomen of a test subject performing training of breathing; a step of detecting displacement of each of the chest and abdomen of the test subject by using the image data; a step of performing control so that movement image indicating movement of each of the chest and abdomen in anteroposterior direction of the test subject is displayed based on the detected displacement of the chest and abdomen, and performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

### Advantageous Effects of Invention

According to the present disclosure, it is possible to provide an information processing apparatus, a display control method, and a program capable of enabling the test subject to easily recognize whether or not the test subject is breathing correctly.

### BRIEF DESCRIPTION OF DRAWINGS

FIG. 1 shows an outline of an information processing apparatus according to an example embodiment of the present disclosure;

FIG. 2 is a flowchart showing a display control method performed by the information processing apparatus according to an example embodiment of the present disclosure;

FIG. 3 shows a display system according to a first example embodiment;

FIG. 4 shows the display system according to the first example embodiment;

FIG. 5 is a flowchart showing a display control method performed by the information processing apparatus according to the first example embodiment;

FIG. 6 shows an example of a photographed image acquired by the image acquisition unit according to the first example embodiment;

FIG. 7 shows an example of a display screen that is displayed on the display device under the control of the display control unit according to the first example embodiment and indicates the displacement in the anteroposterior direction of the chest and the abdomen;

FIG. 8 shows an example of a display screen that is displayed on the display device under the control of the display control unit according to the first example embodiment and indicates the displacement in the anteroposterior direction of the chest and the abdomen;

FIG. 9 shows an example of a display screen that is displayed on the display device under the control of the display control unit according to the first example embodiment and indicates the displacement in the anteroposterior direction of the chest and the abdomen;

FIG. 10 shows an example of a display screen that is displayed on the display device under the control of the display control unit according to the first example embodiment and indicates the displacement in the anteroposterior direction of the chest and the abdomen;

FIG. 11 shows an example of a display screen that is displayed on the display device under the control of the display control unit according to the first example embodiment and indicates the displacement in the anteroposterior direction of the chest and the abdomen;

FIG. 12 shows an example of a display screen that is displayed on the display device under the control of the display control unit according to the first example embodiment and indicates the movement directions in the anteroposterior direction of the chest and the abdomen;

FIG. 13 shows an example of a display screen that is displayed on the display device under the control of the display control unit according to the first example embodiment and indicates the movement directions in the anteroposterior direction of the chest and the abdomen;

FIG. 14 shows an example of a display screen that is displayed on the display device under the control of the display control unit according to the first example embodiment and indicates the movement directions in the anteroposterior direction of the chest and the abdomen;

FIG. 15 shows an example of a display screen that is displayed on the display device under the control of the display control unit according to the first example embodiment and indicates the displacements in the lateral direction of the chest and the abdomen;

FIG. 16 shows an example of an instructor-aimed display screen according to the first example embodiment; and

FIG. 17 shows an example of an instructor-aimed display screen according to the first example embodiment.

### EXAMPLE EMBODIMENT

### (Outline of Example Embodiment According to Present Disclosure)

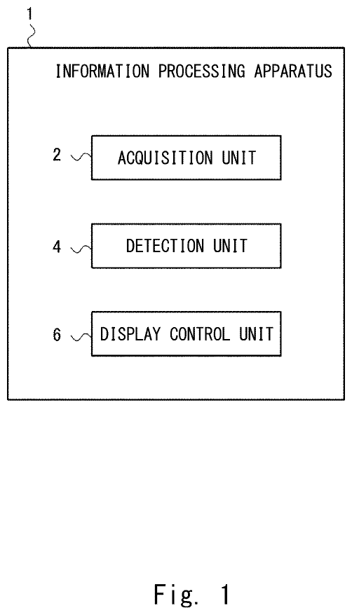

Prior to describing an example embodiment according to the present disclosure, an outline of the example embodiment will be described. FIG. 1 shows an outline of an information processing apparatus 1 according to an example embodiment of the present disclosure. The information processing apparatus 1 is, for example, a computer such as a server or a personal computer.

The information processing apparatus 1 includes an acquisition unit 2, a detection unit 4, and a display control unit 6. The acquisition unit 2 has a function as acquisition means. The detection unit 4 has a function as detection means. The display control unit 6 has a function as display control means.

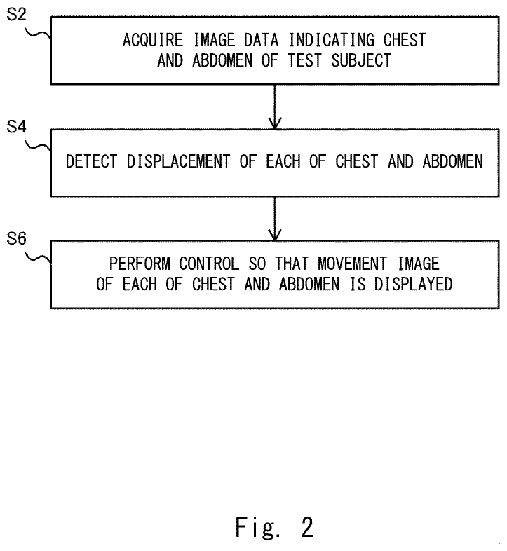

FIG. 2 is a flowchart showing a display control method (an information processing method; a support method) performed by the information processing apparatus 1 according to an example embodiment of the present disclosure. The acquisition unit 2 acquires image data indicating at least a chest and an abdomen of a test subject (Step S2). Here, the “test subject” is a person who performs breathing training. The acquisition unit 2 may acquire image data from, for example, a photographing device (camera) for photographing the test subject. The image data may be, for example, two-dimensional image data such as an RGB image, or three-dimensional image data such as a distance image (three-dimensional point cloud data). Hereinafter, the term “image” also means “image data indicating an image” as a processing object in information processing.

The detection unit 4 detects the displacement of each of the chest and abdomen of the test subject by using the image data (Step S4). The display control unit 6 performs control so that the movement image (i.e., motion image) of each of the chest and abdomen is displayed based on the detected displacement of the chest and abdomen (Step S6). The display control unit 6 may control a display device separate from the information processing apparatus 1 to display the movement image. Alternatively, the display control unit 6 may control a display or the like provided in the information processing apparatus 1 to display the movement image.

Note that the “movement image (i.e., motion image)” is an image indicating the movement of each of the chest and abdomen in the anteroposterior direction of the test subject. Note that the “anteroposterior direction” refers to the anterior (forward) or posterior (backward) direction of the test subject. In other words, when the test subject is in the supine position, the anterior direction corresponds to the upward direction and the posterior direction corresponds to the downward direction. In the following, a movement image showing the movement of the chest in the anteroposterior direction may be referred to as a “chest movement image”. In addition, a movement image showing the movement of the abdomen in the anteroposterior direction may be referred to as an “abdomen movement image (i.e., abdominal movement image)”.

Here, when the movement of the chest and the movement of the abdomen are synchronized with each other, the display control unit 6 performs control so that the display form of the chest movement image and the display form of the abdomen movement image are the same as each other. When the movement of the chest and the movement of the abdomen are not synchronized with each other, the display control unit 6 performs control so that the display form of the chest movement image and the display form of the abdomen movement image are different from each other. Here, “same display form” means, for example, the same color representation, but it is not limited to this. In other words, “display form” is not limited to “color representation”.

For example, in the case where the display form is “color representation”, the display control unit 6 performs control so that the color representation of the chest movement image and the color representation of the abdomen movement image are the same as each other when the movement of the chest and the movement of the abdomen are synchronized with each other. The display control unit 6 performs control so that the color representation of the chest movement image and the color representation of the abdomen movement image are different from each other when the movement of the chest and the movement of the abdomen are not synchronized with each other.

Note that the “movement (i.e., motion)” may be defined by, for example, the displacement of each of the chest and the abdomen in the anteroposterior direction relative to the reference position. In this case, for example, the displacement of the chest may be a positive (+) value (a value greater than or equal to 0) when the position of the chest is on the anterior side of the reference position (the chest reference position), or a negative (−) value (a value less than 0) when the position of the chest is on the posterior side of the reference position. Similarly, for example, the displacement of the abdomen may be a positive (+) value (a value greater than or equal to 0) when the position of the abdomen is on the anterior side of the reference position (the abdomen reference position), or a negative (−) value (a value less than 0) when the position of the abdomen is on the posterior side of the reference position. The reference position will be described later.

In addition, the “movement” may be defined, for example, in the direction of movement (i.e., “movement direction”) of the chest and abdomen, respectively. Here, the “movement direction” is the direction of change (increase or decrease) of the displacement of the chest and abdomen, respectively. In this case, for example, the movement direction of the chest may be in the anterior direction (forward direction; positive direction) when the displacement of the chest increases (e.g., in the inspiratory state) and in the posterior direction (backward direction; negative direction) when the displacement of the chest decreases (e.g., in the expiratory state). Similarly, for example, the direction of movement of the abdomen may be in the forward direction (anterior direction; positive direction) when the displacement of the abdomen increases (e.g., in the inspiratory state) and in the backward direction (posterior direction; negative direction) when the displacement of the abdomen decreases (e.g., in the expiratory state).

As described above, in order to improve and maintain the state of health, it is desirable to perform breathing with the correct breathing technique. In order to perform correct breathing, it is desirable to continuously perform correct breathing training based on the guidance of a training instructor such as a therapist (hereinafter simply referred to as “instructor”). For example, correct breathing training can improve health conditions such as physical functions, such as back pain, and mental status.

Here, in the breathing training, it is considered that the effect of the training is favorable if the test subject performs breathing so as to satisfy the condition that the anteroposterior movement (motion) of the chest and the anteroposterior movement (motion) of the abdomen are synchronized with each other (“synchronization between the chest and the abdomen”). In addition, it is considered that the effect of the training is favorable if the test subject performs breathing so as to satisfy the condition that the rib is sufficiently internally rotated during expiration (exhalation), that is, the condition that the lateral displacement of the chest is sufficiently small during exhalation; “rib internal-rotation”). However, it is difficult for the test subject to confirm the above. That is, it is difficult for the test subject to recognize his/her breathing condition.

In contrast, since the information processing apparatus 1 according to the present disclosure is configured as described above, it can cause the chest movement image and the abdomen movement image to be displayed so that it is easy to visually understand whether the chest movement and the abdomen movement are synchronized with each other. Therefore, the information processing apparatus 1 according to the present disclosure enables the test subject to easily recognize whether or not the test subject is breathing correctly.

Note that the test subject can easily recognize whether or not the test subject is breathing correctly by using a display system including a photographing device, a display device, and the information processing apparatus 1. In addition, the test subject can easily recognize whether or not the test subject is breathing correctly by using a display control method performed by the information processing apparatus 1 or a program for performing the display control method.

### First Example Embodiment

An example embodiment will be described hereinafter with reference to the drawings. For clarifying the explanation, the following description and the drawings have been partially omitted and simplified as appropriate. Further, the same symbols are assigned to the same or corresponding components throughout the drawings and redundant descriptions thereof are omitted as appropriate.

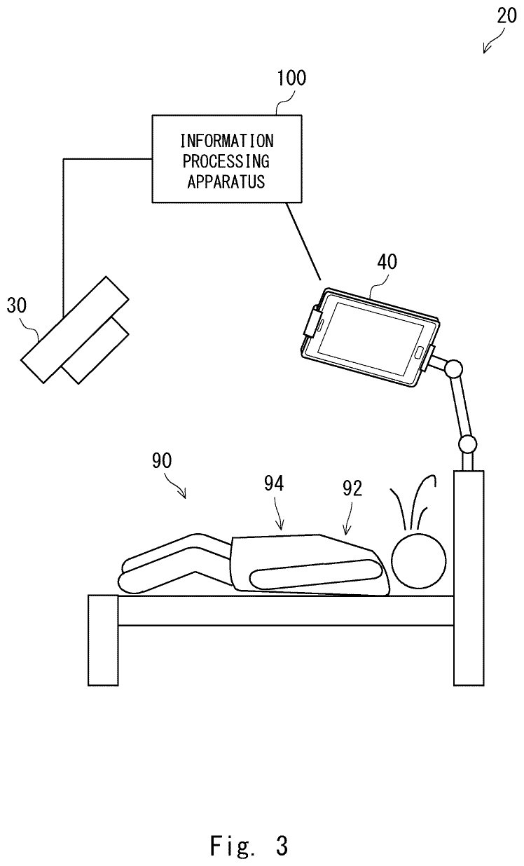

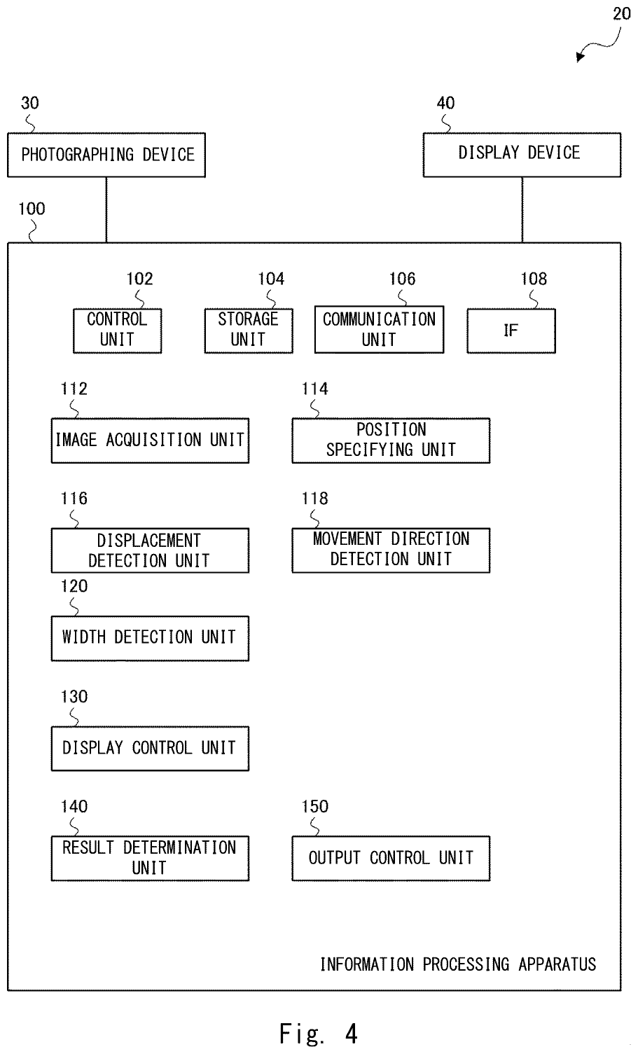

FIGS. 3 and 4 shows a display system 20 according to a first example embodiment. FIG. 3 illustrates a state in which the display system 20 is used for breathing training of a test subject 90. FIG. 4 is a functional block diagram showing a configuration of the display system 20. As shown in FIG. 3, although the test subject 90 preferably performs breathing training in the supine position, the position of the test subject 90 is not limited to the supine position. In the example embodiment described below, the test subject 90 performs breathing training in the supine position.

The display system 20 includes at least one photographing device 30, a display device 40, and an information processing apparatus 100. The information processing apparatus 100 corresponds to the information processing apparatus 1 shown in FIG. 1. The information processing apparatus 100 is communicatively connected to the photographing device 30 and the display device 40 through a wired or wireless network so that they can communicate with each other.

The photographing device 30 photographs the test subject 90 performing breathing training. The photographing device 30 may be installed on the position in which a chest 92 and an abdomen 94 of the test subject 90 can be photographed. When the test subject 90 performs breathing training in the supine position, the photographing device 30 may be installed, for example, over the chest 92 and abdomen 94 of the test subject 90. That is, the photographing device 30 may be located in a position opposed to the test subject 90 in the supine position. The test subject 90 may perform breathing training while wearing clothing. In this case, the chest 92 is a part corresponding to the chest of the test subject 90 while wearing clothing. Similarly, the abdomen 94 is a part corresponding to the abdomen of the test subject 90 while wearing clothing.

The photographing device 30 is, for example, a camera. The photographing device 30 may be a two-dimensional camera (for example, an RGB camera, etc.), a three-dimensional camera (for example, a depth sensor, a LiDAR (Light Detection and Ranging), a stereo camera, etc.), or a camera including both of these (for example, an RGB-D camera, etc.). The photographing device 30 may measure the distance to the photographed object by, for example, a ToF (Time of Flight) method.

By using the photographing device 30, the position of the test subject 90 can be detected. The movement of the test subject 90 can be detected by using the photographing device 30. For example, motion capture and the like can be realized by using the photographing device 30. Furthermore, skeleton data indicating the skeletal frame (joint) of the photographed test subject 90 may be generated by using the photographing device 30. The skeleton data is data showing the position of the joint of the test subject 90. The skeleton data may be acquired, for example, by the photographing device 30 (or the information processing apparatus 100) by recognizing the joint of a moving person.

The photographing device 30 generates image data showing at least the chest 92 and abdomen 94 of the test subject 90 by photographing the test subject 90. That is, the image data may indicate an image (photographed image) showing the chest 92 and abdomen 94 of the test subject 90 and their surroundings. The photographed image may be moving images or still images. As described above, the image data may be, for example, two-dimensional image data such as RGB images or three-dimensional image data such as distance images (three-dimensional point cloud data). Alternatively, the image data may be data indicating an image obtained by combining the two-dimensional image and the three-dimensional image. Accordingly, the image data may indicate three-dimensional coordinates (position information) of the position of the surface of the photographed test subject 90, by three-dimensional point cloud data or the like. The image data may also include the skeleton data described above. The photographing device 30 transmits the generated image data to the information processing apparatus 100.

By using the image data, the position of the chest 92 and the position of the abdomen 94 of the test subject 90 can be detected. By using the image data, the movement of the chest 92 and the movement of the abdomen 94 of the test subject 90 can be detected. Thus, the photographing device 30 can also function as a detection device capable of detecting the position (and movement) of the test subject 90.

The information processing apparatus 100 acquires image data from the photographing device 30. The information processing apparatus 100 detects displacement of each of the chest 92 and abdomen 94 of the test subject 90 by using the acquired image data. Further, the information processing apparatus 100 performs control so that the movement images of each of the chest 92 and abdomen 94 (“chest movement image” and “abdomen movement image”) are displayed based on the detected displacement the chest 92 and abdomen 94. Details will be described later.

The display device 40 displays movement images of the chest 92 and the abdomen 94, respectively, i.e., “chest movement image” and “abdomen movement image”, under the control of the information processing apparatus 100. The display device 40 displays information about the training of the test subject 90. Preferably, the display device 40 displays an image for the test subject 90. The display device 40 may display an image for the test subject 90 when it is placed above the test subject 90. For example, the display device 40 may display an image for the test subject 90 when a camera built into the display device 40 detects the face of the test subject 90.

The display device 40 is disposed so as to display the image in a position visible to the test subject 90. The display device 40 includes, for example, a display for displaying the image. The display device 40 includes, for example, but is not limited to, a liquid crystal display (LCD). The display device 40 may be implemented by an organic EL (Electro-Luminescence) display, a projector, or the like. The display device 40 may be, for example, a smartphone or a tablet terminal. Details of the contents displayed by the display device 40 will be described later.

FIG. 4 shows a configuration of the information processing apparatus 100 according to the first example embodiment. As shown in FIG. 4, the information processing apparatus 100 includes, as its main hardware configuration, a control unit 102, a storage unit 104, a communication unit 106, and an interface (IF) unit 108. The control unit 102, the storage unit 104, the communication unit 106, and the interface unit 108 are connected to each other through a data bus or the like. The photographing device 30 and the display device 40 may also include the hardware configuration of the information processing apparatus 100 shown in FIG. 4.

The control unit 102 is, for example, a processor such as a CPU (Central Processing Unit). The control unit 102 functions as an arithmetic apparatus that performs control processing, arithmetic processing, and the like. The storage unit 104 is a storage device such as a memory or a hard disk drive. The storage unit 104 is, for example, a ROM (Read Only Memory) or a RAM (Random Access Memory). The storage unit 104 has a function for storing a control program(s), an arithmetic program(s), and the like that are executed by the control unit 102. Further, the storage unit 104 has a function for temporarily storing processing data and the like. The storage unit 104 may include a database.

The communication unit 106 performs processing necessary to communicate with the photographing device 30 and the display device 40 (and other devices) through a network. The communication unit 106 can include communication ports, a router, a firewall, and the like. The interface (IF: Interface) unit 108 is, for example, a user interface (UI). The interface unit 108 includes an input device such as a keyboard, a touch panel, or a mouse, and an output device such as a display or a speaker(s). The interface unit 108 receives data input by a user (an operator) and outputs information for the user. The interface unit 108 may display information about the breathing training of the test subject 90. For example, the interface unit 108 may display an image for an instructor. The image for the instructor may include, for example, a setting screen for breathing training.

The information processing apparatus 100 according to the first example embodiment includes, as its components, an image acquisition unit 112, a position specifying unit 114, a displacement detection unit 116, a movement direction detection unit 118, a width detection unit 120, a display control unit 130, a result determination unit 140, and an output control unit 150. The image acquisition unit 112 corresponds to the acquisition unit 2 shown in FIG. 1. The image acquisition unit 112 has a function as image acquisition means (acquisition means). The position specifying unit 114 has a function as position specifying means. The displacement detection unit 116 corresponds to the detection unit 4 shown in FIG. 1. The displacement detection unit 116 has a function as displacement detection means (detection means and calculation means). The movement direction detection unit 118 has a function as movement direction detection means (detection means and calculation means). The width detection unit 120 has a function as width detection means. The display control unit 130 corresponds to the display control unit 6 shown in FIG. 1. The display control unit 130 has a function as display control means. The result determination unit 140 has a function as result determination means (determination means). The output control unit 150 has a function as output control means. The specific functions of each component will be described later.

Note that each of the above-described components can be implemented, for example, by executing a program under the control of the control unit 102. More specifically, each of the components can be implemented by having the control unit 102 execute a program stored in the storage unit 104. Further, each of the components can be implemented by recording a necessary program on an arbitrary non-volatile recording medium and installing it as required. Further, each of the components is not limited to those implemented by software using a program, and may be implemented by a combination of any two or more of hardware, firmware, and software. Further, each of the components may be implemented by using a user-programmable integrated circuit such as an FPGA (Field-Programmable Gate Array) or a microcomputer. In such a case, a program composed of a respective one of the above-described components may be implemented by using this integrated circuit.

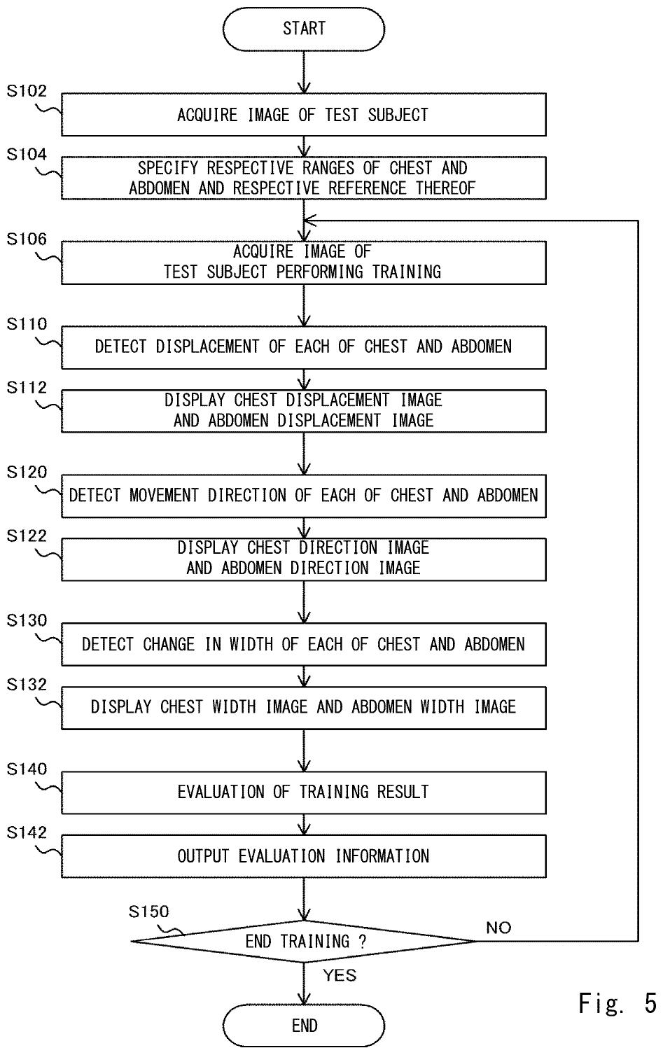

FIG. 5 is a flowchart showing a display control method performed by the information processing apparatus 100 according to the first example embodiment. Note that the display control method can be said to be an information processing method for performing display control. The display control method can also be said to be a support method for performing display for supporting breathing training to a user such as the test subject 90.

The image acquisition unit 112 acquires the image of the test subject 90 (Step S102). The image acquisition unit 112 acquires (receives) the image data indicating at least the chest 92 and abdomen 94 of the test subject 90 from the photographing device 30 by using the communication unit 106.

In the process of S102, the image acquisition unit 112 acquires the image of the test subject 90 in the resting state. Specifically, the instructor instructs the test subject 90 to relax and rest, and the photographing device 30 photographs the test subject 90 in the resting state. As a result, the image acquisition unit 112 acquires the image of the test subject 90 in a normal state, that is, when the test subject is breathing at rest. As described above, the image of the test subject 90 may be a moving image or a still image.

The position specifying unit 114 specifies the respective ranges of the chest 92 and the abdomen 94 in the acquired image and the respective reference of the chest 92 and the abdomen 94 (Step S104). Specifically, the position specifying unit 114 specifies the region (chest region) corresponding to the chest 92 in the acquired image. Similarly, the position specifying unit 114 specifies the region (abdomen region) corresponding to the abdomen 94 in the acquired image. Thus, the regions corresponding to the chest 92 and the abdomen 94 in the image acquired during the breathing training can be specified.

Regarding specifying the region corresponding to the chest 92, the position specifying unit 114 may specify the region corresponding to the chest 92 in the image by using, for example, skeleton data included in the image data. The position specifying unit 114 may specify the region corresponding to the chest 92 in the image by using, for example, a learned model learned by machine learning. The learned model is learned so that an image of a test subject is used as an input and the region of the chest in the image is output from it. The position specifying unit 114 may specify the region corresponding to the chest 92 by, for example, an operation by a user such as an instructor. In this case, the user may select the region corresponding to the chest 92 on the image of the test subject 90 displayed on the touch panel by, for example, tracing the region corresponding to the chest 92 on the touch panel with a finger. The position specifying unit 114 may specify the region corresponding to the abdomen 94 in substantially the same manner as described above. For example, the position specifying unit 114 may specify the region corresponding to the sternum (and its surroundings) of the test subject 90 as the chest region. The position specifying unit 114 may specify the region corresponding to the umbilicus (and its surroundings) of the test subject 90 as the abdomen region.

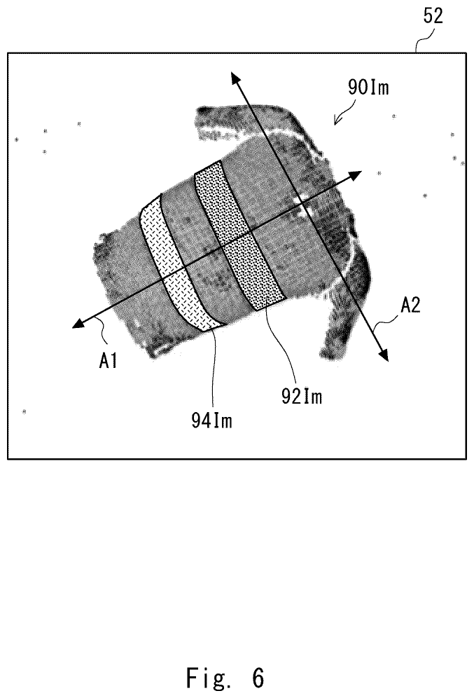

FIG. 6 shows an example of a photographed image 52 acquired by the image acquisition unit 112 according to the first example embodiment. Here, when the photographed image 52 has three-dimensional information, the pixels constituting the photographed image 52 may include position information (distance information) of the parts of a photographic subject corresponding to the pixels. The photographed image 52 includes the test subject image 901m, which is an image of the test subject 90. In the test subject image 901m of the photographed image 52, a chest region 921m and an abdomen region 941m specified by the position specifying unit 114 are shown. In the captured image 52, for example, the test subject image 901m may be shown in red, the chest region 921m may be shown in green, and the abdomen region 941m may be shown in blue.

The position specifying unit 114 may specify the anteroposterior direction and the lateral direction of the test subject 90. For example, the position specifying unit 114 may specify the anteroposterior direction and the lateral direction of the test subject 90 by using skeleton data. Alternatively, the position specifying unit 114 may recognize the head and the lower limb of the test subject 90 and specify the upper-lower direction of the test subject 90 from the direction of the center line of the test subject 90 (arrow A1 in FIG. 6) recognized from the recognized head and the recognized lower limb. In addition, the position specifying unit 114 may recognize both shoulders of the test subject 90 and specify the lateral direction of the test subject 90 from the direction of the line connecting the recognized shoulders (arrow A2 in FIG. 6). The position specifying unit 114 may specify a direction perpendicular to the specified upper-lower direction and the specified lateral direction, as the anteroposterior direction. The position specifying unit 114 may recognize the face of the test subject 90 and specify the recognized face direction, as the anterior direction. Alternatively, when the test subject 90 is in a supine position on a horizontal surface, the position specifying unit 114 may specify the direction along the vertical direction, as the anteroposterior direction.

In addition, the position specifying unit 114 specifies the reference positions of the chest 92 and the abdomen 94, respectively, by using the photographed image 52 acquired when the test subject 90 is in a resting state. Specifically, the position specifying unit 114 specifies the reference position of the chest 92 by using position information corresponding to the chest region specified in the photographed image 52 in a resting state. Similarly, the position specifying unit 114 specifies the reference position of the abdomen 94 by using position information corresponding to the abdomen region specified in the photographed image 52 in a resting state. The position specifying unit 114 may specify the reference position of the torso (trunk) including the chest 92 and the abdomen 94 of the test subject 90.

Here, the position specifying unit 114 specifies the reference positions of the chest 92 and the abdomen 94 in the anteroposterior direction. The position specifying unit 114 specifies the chest reference position which is the reference position of the chest 92 in the anteroposterior direction. Similarly, the position specifying unit 114 specifies the abdomen reference position which is the reference position of the abdomen 94 in the anteroposterior direction. For example, the chest reference position may be the average position of the surface (front) of the chest 92 in the anteroposterior direction between expiration and inspiration in a resting state. Similarly, the abdomen reference position may be the average position of the surface (front) of the abdomen 94 in the anteroposterior direction between expiration and inspiration in a resting state. The position specifying portion 114 may specify the reference position of the trunk including the chest 92 and the abdomen 94 in the anteroposterior direction (the torso reference position).

Here, the chest reference position may be, for example, the average anteroposterior position of the positions (corresponding to the “height” in the case of the supine position) at each position on the entire surface of the chest 92 at rest (the first chest reference position). In this case, the chest reference position may be indicated by one value. Alternatively, the chest reference position may be, for example, anteroposterior position(s) of one or more specific points on the surface of the chest 92 (e.g., a point in the middle of the sternum of the chest 92) at rest (the second chest reference position). In this case, the chest reference position may be indicated by the number (N) of values corresponding to the number of specific points. Alternatively, the chest reference position may be, for example, anteroposterior position(s) of M points arranged at predetermined intervals in the upper-lower direction (corresponding to arrow A1 in FIG. 6) on the surface of the chest 92 at rest (the third chest reference position). In this case, the chest reference position may be indicated by M values. In the third chest reference position, each of the “anteroposterior position(s) of M points arranged at predetermined intervals in the upper-lower direction” may be the average anteroposterior position (or the most anterior position) in the areas separated by dividing the chest region at predetermined intervals in the upper-lower direction. In addition, the chest reference position may be the anteroposterior positions of n points on the entire surface of the chest 92 at rest (the fourth chest reference position). The same applies to the abdomen reference position and the torso reference position.

The position specifying unit 114 specifies the reference positions (reference widths) in the lateral direction (width direction) of the chest 92 and the abdomen 94, respectively, by using the photographed image 52 acquired when the test subject 90 is in a resting state. The position specifying unit 114 specifies the chest reference width which is the reference width in the lateral direction of the chest 92. Similarly, the position specifying unit 114 specifies the abdomen reference width which is the reference position in the lateral direction of the abdomen 94. For example, the chest reference width may be the average width of the chest 92 between expiration and inspiration in a resting state. Similarly, the abdomen reference width may be the average width of the abdomen 94 between expiration and inspiration in a resting state. Here, the chest reference width may be, for example, the distance between the left and right ends of the chest 92 in a resting state. Similarly, the abdomen reference width may be, for example, the distance between the left and right ends of the abdomen 94 in a resting state.

The position specifying unit 114 may set a threshold Th1 of the width of the chest 92 at the time of expiration. The threshold Th1 corresponds to the width of the chest 92 when he/she fully exhales at the time of expiration. Therefore, if the width of the chest 92 decreases to the threshold Th1 at the time of expiration, it can be said that the rib internal-rotation is sufficiently performed. Therefore, it can be said that the threshold Th1 is the target value of the chest width at the time of expiration. The threshold Th1 is appropriately set by the instructor who has confirmed the respiratory condition of the test subject 90. The threshold Th1 is smaller than the chest reference width. Therefore, when the chest reference width is set to 0, the threshold Th1 becomes a negative value.

The image acquisition unit 112 acquires an image of the test subject 90 performing breathing training (Step S106). Specifically, the instructor instructs the test subject 90 to perform breathing training, and the photographing device 30 photographs the test subject 90 in that state. Thus, the image acquisition unit 112 acquires the image of the test subject 90 performing breathing training. The image acquisition unit 112 preferably acquires a moving image of the test subject 90 performing breathing training. Thus, as will be described later, the information processing apparatus 100 can detect changes in the displacements of the chest 92 and the abdomen 94.

The processing of S106 and S110 to S132 described later can be executed for each frame of the acquired moving image. That is, while the test subject 90 is performing breathing training, the processing of S106 to S132 is continuously executed. In other words, the processing of S110 to S132 is executed for one frame of the moving image obtained by photographing the test subject 90 performing breathing training, and when the next frame is acquired, the processing of S110 to S132 is executed for the next frame. If necessary, the processing of S140 to S142 described later may also be executed for each frame.

The displacement detection unit 116 detects the displacement from the reference position for each of the chest 92 and the abdomen 94 (Step S110). Specifically, the displacement detection unit 116 detects a change in the anteroposterior position (corresponding to the “height” in the supine position) of each of the chest 92 and the abdomen 94. More specifically, the displacement detection unit 116 specifies the chest region and the abdomen region in the photographed image 52 of the test subject 90 acquired in the process of S106. This specifying process may be performed by using the process of specifying the regions corresponding to the chest 92 and the abdomen 94, respectively, in S104.

In addition, the displacement detection unit 116 detects the anteroposterior positions of the chest 92 and the abdomen 94, respectively (corresponding to the “height” in the case of the supine position) in the same manner as when the reference positions of the chest 92 and the abdomen 94, respectively, in the anteroposterior direction are specified in the process of S104. Specifically, the displacement detection unit 116 detects the anteroposterior position of the chest 92 (the chest position) by using the position information corresponding to the chest region specified in the photographed image 52 acquired in the process of S106. Similarly, the displacement detection unit 116 detects the anteroposterior position of the abdomen 94 (the abdomen position) by using the position information corresponding to the abdomen region specified in the photographed image 52 acquired in the process of S106.

Furthermore, the displacement detection unit 116 calculates the displacement of the chest position relative to the chest reference position specified in S104. The displacement detection unit 116 calculates the difference between the chest position and the chest reference position as the displacement (chest displacement). Here, when the chest position is more anterior than the chest reference position, that is, when the chest 92 is more expanded than that at rest, the sign of the displacement is positive (+). On the other hand, when the chest position is more posterior than the chest reference position, that is, when the chest 92 is more contracted than that at rest, the sign of the displacement is negative (−). Therefore, the displacement detection unit 116 calculates the displacement of the chest 92 by subtracting the value of the chest reference position from the value of the chest position.

The displacement detection unit 116 calculates the displacement of the abdomen position relative to the abdomen reference position specified in S104. The displacement detection unit 116 calculates the difference between the abdomen position and the abdomen reference position as the displacement (abdomen displacement). Here, when the abdomen position is more anterior than the abdomen reference position, that is, when the abdomen 94 is more expanded than that at rest, the sign of the displacement is positive (+). On the other hand, when the abdomen position is more posterior than the abdomen reference position, that is, when the abdomen 94 is more contracted than at rest, the sign of the displacement is negative (−). Therefore, the displacement detection unit 116 calculates the displacement of the abdomen 94 by subtracting the value of the abdomen reference position from the value of the abdomen position.

The displacement detection unit 116 may detect the displacement in the anteroposterior direction of the torso including the chest 92 and the abdomen 94 in the same manner as the chest 92 and the abdomen 94. In this case, the displacement detection unit 116 detects the torso position, which is the anteroposterior position of the torso (corresponding to the “height” in the supine position), and calculates the difference obtained by subtracting the torso reference position from the detected torso position as the displacement of the torso (torso displacement).

When the chest reference position is the average position of the entire surface of the chest 92 at rest (the first chest reference position described above), the displacement detection unit 116 calculates the average position of the anteroposterior positions at the respective points on the surface of the chest 92, as the chest position (the first chest position). The displacement detection unit 116 calculates the difference between the calculated first chest position and the first chest reference position. When the chest reference position is the anteroposterior position(s) of the specific point(s) on the surface of the chest 92 at rest (the second chest reference position described above), the displacement detection unit 116 detects the anteroposterior position(s) of the specific point(s) (the second chest position) in the chest region specified by the processing in S110. The displacement detection unit 116 calculates the difference (displacement) between the detected second chest position and the second chest reference position of the corresponding point. In this case, the displacement detection unit 116 may further calculate the sum or average value of the N differences obtained for each of the N specified points as the displacement. When the chest reference position is the third chest reference position described above, the displacement detection unit 116 calculates the anteroposterior position of each of the M points arranged at predetermined intervals in the upper-lower direction of the chest region specified in the process of S110 as the chest position (third chest position). The displacement detection unit 116 calculates the difference (displacement) between the calculated third chest position and the corresponding third chest reference position. In this case, the displacement detection unit 116 may further calculate the sum or average value of the M differences obtained for each of the M calculated points as the displacement. When the chest reference position is the fourth chest reference position described above, the displacement detection unit 116 calculates the anteroposterior position of each of the n points of the entire chest region specified in the process of S110 as the chest position (fourth chest position). The displacement detection unit 116 calculates the difference (displacement) between the fourth chest position and the fourth chest reference position of the corresponding point. Furthermore, the displacement detection unit 116 may calculate the sum of the calculated n differences (displacement) as the sum of the displacements. The same applies to the abdomen displacement and the torso displacement.

The display control unit 130 performs control for displaying the displacement image (Step S112). The displacement image is the movement image described above and is an image indicating the displacement in the anteroposterior direction. The display control unit 130 performs control for displaying a chest displacement image and an abdomen displacement image. Here, the “chest displacement image” is the chest movement image described above and is an image indicating the displacement of the chest 92 from the chest reference position (displacement image). That is, the chest displacement image is an image indicating the displacement of the chest position relative to the chest reference position (chest displacement). The “abdomen displacement image” is the abdomen movement image described above and an image indicating the displacement of the abdomen 94 from the abdomen reference position (displacement image). That is, the abdomen displacement image is an image indicating the displacement of the abdomen position relative to the abdomen reference position (abdomen displacement).

Here, when the chest displacement and the abdomen displacement are synchronized with each other, the display control unit 130 performs control so that the display form of the chest displacement image and the display form of the abdomen displacement image are the same as each other. On the other hand, when the chest displacement and the abdomen displacement are not synchronized with each other, the display control unit 130 performs control so that the display form of the chest displacement image and the display form of the abdomen displacement image are different from each other.

Here, the case where “the chest displacement and the abdomen displacement are synchronized with each other” is a case where the sign (positive or negative) of the chest displacement and the sign (positive or negative) of the abdomen displacement are the same as each other. Conversely, the case where “the chest displacement and the abdomen displacement are not synchronized with each other” is a case where the sign (positive or negative) of the chest displacement and the sign (positive or negative) of the abdomen displacement are different from each other. Therefore, when the sign of the chest displacement and the sign of the abdomen displacement are the same as each other, the display control unit 130 performs control so that the display form of the chest displacement image and the display form of the abdomen displacement image are the same as each other. On the other hand, when the sign of the chest displacement and the sign of the abdomen displacement are different from each other, the display control unit 130 performs control so that the display form of the chest displacement image and the display form of the abdomen displacement image are different from each other.

The “display form” is, for example, a color representation. In this case, when the chest displacement and the abdomen displacement are synchronized with each other, the display control unit 130 performs control so that the color representation of the chest displacement image and the color representation of the abdomen displacement image are the same as each other. That is, when the sign of the chest displacement and the sign of the abdomen displacement are the same as each other, the display control unit 130 performs control so that the color representation of the chest displacement image and the color representation of the abdomen displacement image are the same as each other. On the other hand, when the sign of the chest displacement and the sign of the abdomen displacement are different from each other, the display control unit 130 performs control so that the color representation of the chest displacement image and the color representation of the abdomen displacement image are different from each other. Note that the display form is not limited to the color representation. In the first example embodiment, the “display form” is assumed to be the color representation.

For example, the display control unit 130 performs control so that, for each of the chest displacement image and the abdomen displacement image, the displacement is displayed in red when the sign of the displacement is “positive”. Therefore, when the sign of the chest displacement is “positive”, the chest displacement image is displayed in red. Similarly, when the sign of the abdomen displacement is “positive”, the abdomen displacement image is displayed in red. On the other hand, the display control unit 130 performs control so that, for each of the chest displacement image and the abdomen displacement image, the displacement is displayed in blue when the sign of the displacement is “negative”. Therefore, when the sign of the chest displacement is “negative”, the chest displacement image is displayed in blue. Similarly, when the sign of the abdomen displacement is “negative”, the abdomen displacement image is displayed in blue.

The display control unit 130 generates data indicating an instruction for displaying the displacement image (displacement image display instruction data) and transmits the data to the display device 40. The displacement image display instruction data may indicate the displacement and a color representation to be displayed according to the sign of the displacement. In particular, the displacement image display instruction data may indicate a color representation to be displayed according to the chest displacement and the sign of the chest displacement, and a color representation to be displayed according to the abdomen displacement and the abdomen displacement. In addition, the displacement image display instruction data may indicate a color representation to be displayed according to the torso displacement and the sign of the torso displacement. Thus, the display device 40 displays the displacement image in a color representation according to the sign of the displacement.

The display control unit 130 performs control so that the displacement image is displayed so as to change in real time in accordance with the progress of the breathing training. That is, the display control unit 130 causes the display device 40 to display the displacement image that changes according to the progress of the breathing training of the test subject 90. In other words, the display control unit 130 performs control so that the displacement image (movement image) that changes in accordance with the progress of the breathing training of the test subject 90 is displayed during the progress of the breathing training. In other words, the display control unit 130 performs control so that the displacement image corresponding to the displacement at a certain timing during the breathing training is displayed at that timing. For example, when the displacement is detected at time t1, the displacement image may be displayed at time t1+Δt. Here, Δt is a minute time. Δt can be determined according to the processing capacity of the information processing apparatus 100 and the display device 40 and the communication environment between the information processing apparatus 100 and the display device 40.

The display control unit 130 generates the displacement image display instruction data every time each frame of the photographed image of the test subject 90 performing the breathing training is acquired, and transmits the data to the display device 40. Accordingly, the display control unit 130 can cause the display device 40 to display the displacement image that changes with the progress of the breathing training.

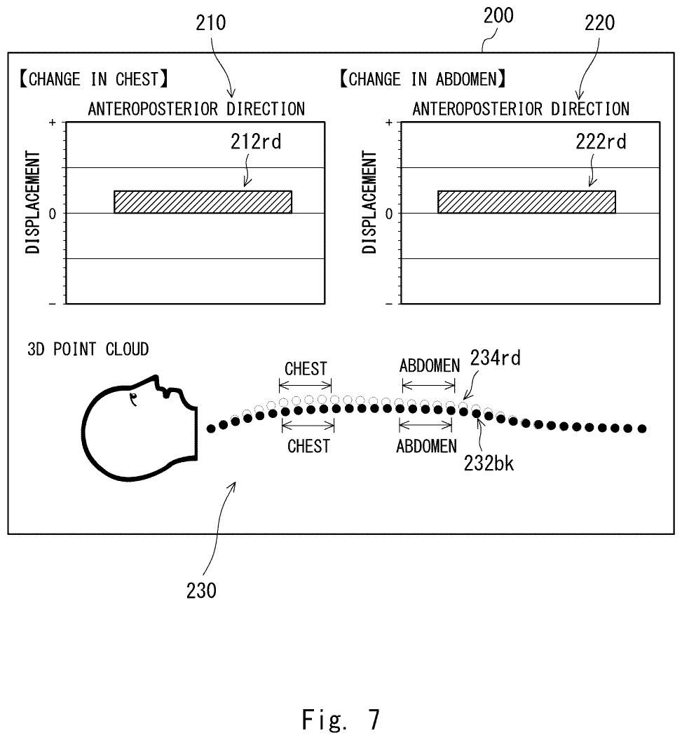

FIGS. 7 to 11 show examples of display screens 200 that are displayed on the display device 40 under the control of the display control unit 130 according to the first example embodiment and indicate the displacement in the anteroposterior direction of the chest 92 and the abdomen 94. FIG. 7 shows the display screen 200 in a case where the sign of the displacement in the anteroposterior direction of the chest 92 (chest displacement) and the sign of the displacement in the anteroposterior direction of the abdomen 94 (abdomen displacement) are both positive. FIG. 7 shows the display screen 200 displaying the displacement at a certain timing during breathing training. The display screen 200 shown in FIG. 7 is displayed on the display device 40 at that timing. That is, the display screen 200 shown in FIG. 7 is displayed on the display device 40 in real time during breathing training.

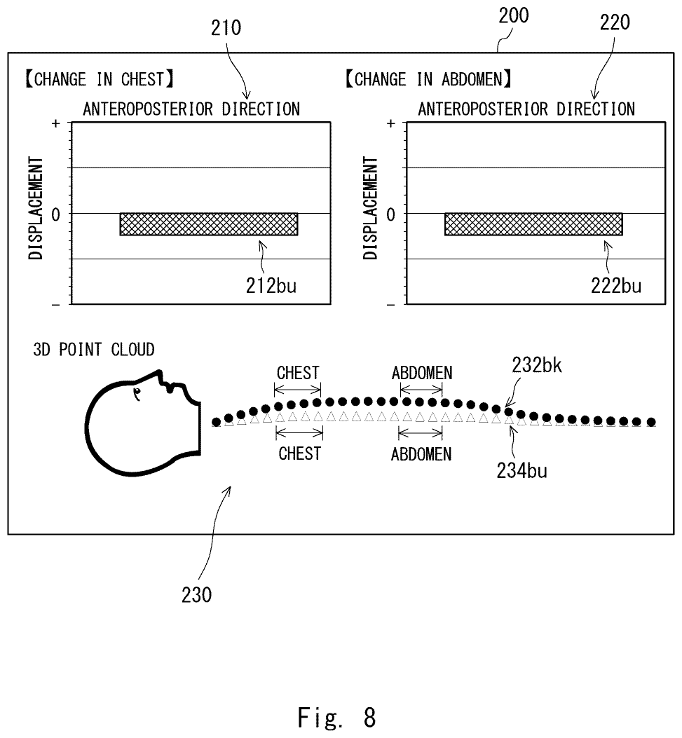

The display screen 200 shown in FIG. 7 displays the chest displacement screen 210, the abdomen displacement screen 220, and the torso displacement screen 230. The display screen 200 may display only the chest displacement screen 210 and the abdomen displacement screen 220 without displaying the torso displacement screen 230. Alternatively, the display screen 200 may display only the torso displacement screen 230 without displaying the chest displacement screen 210 and the abdomen displacement screen 220. The same applies to the case in FIG. 8 and the like, which will be described below.

In FIG. 7, the chest displacement screen 210 displays a chest displacement image 212rd indicating that the sign of the chest displacement is positive. The abdomen displacement screen 220 displays an abdomen displacement image 222rd indicating that the sign of the abdomen displacement is positive. Here, the chest displacement image 212rd and the abdomen displacement image 222rd are drawn in red, which is the same color representation. The vertical length of each of the chest displacement image 212rd and the abdomen displacement image 222rd indicates a scalar value of the displacement. That is, the chest displacement image 212rd indicates the chest displacement at the timing when the display screen 200 (the chest displacement image 212rd) is displayed. Similarly, the abdomen displacement image 222rd indicates the abdomen displacement at the timing when the display screen 200 (the abdomen displacement image 222rd) is displayed.

Here, the chest displacement and the abdomen displacement change with the passage of time. Therefore, in the display screen 200, the chest displacement image 212 and the abdomen displacement image 222 change with the passage of time. The same applies to the case in FIG. 8 and the like.

Here, in the example of FIG. 7, since the sign of the chest displacement and the sign of the abdomen displacement are both positive, the chest displacement and the abdomen displacement are synchronized with each other. The chest displacement image 212rd and the abdomen displacement image 222rd are both drawn in red. Therefore, in the example of FIG. 7, the chest displacement image 212rd and the abdomen displacement image 222rd are displayed so that it can be easily recognized visually that the chest displacement and the abdomen displacement are synchronized with each other. Therefore, the test subject 90 who sees the display screen 200 shown in FIG. 7 can easily recognize that the movement of the chest 92 and the movement of the abdomen 94 are synchronized with each other. Therefore, the test subject 90 can easily recognize his/her own breathing condition.

In FIG. 7, the torso displacement screen 230 displays a set of points (dot row) corresponding to a three-dimensional point cloud of the torso of the test subject 90 during breathing training. In the torso displacement screen 230, the upward direction in FIG. 7 corresponds to the anterior direction of the test subject 90, and the downward direction corresponds to the posterior direction of the test subject 90. The torso displacement screen 230 displays the range corresponding to the chest 92 and the range corresponding to the abdomen 94 of the test subject 90.

In FIG. 7, a torso reference position dot row 232bk is shown as a dot row composed of a plurality of dots each formed by a black circle. The torso reference position dot row 232bk indicates the torso reference position. In the display screen 200, the torso reference position dot row 232bk is drawn in black. Each of dots constituting the torso reference position dot row 232bk indicates a position in the anteroposterior direction (corresponding to “height” in the supine position) at each point, corresponding to the dot, in the upper-lower direction (the lateral direction in FIG. 7) of the test subject 90, on the torso surface of the test subject 90 at rest.

Further, in FIG. 7, the torso position dot row 234rd is shown as a dot row composed of a plurality of dots each formed by an open circle. Each of dots constituting the torso position dot row 234rd indicates a position in the anteroposterior direction (corresponding to a “height” in the supine position) at each point, corresponding to the dot, in the upper-lower direction (the lateral direction in FIG. 7) of the test subject 90, on the torso surface of the test subject 90 during training. In other words, the torso position dot row 234rd indicates a torso displacement at the timing when the display screen 200 (the torso position dot row 234rd) is displayed. That is, the torso position dot row 234rd corresponds to the displacement image described above. The torso position dot row 234rd is drawn in red when the torso position is located ahead the torso reference position (that is, when the torso displacement is positive).

Here, the torso displacement changes with the passage of time. Therefore, in the display screen 200, the torso position dot row 234 changes with the passage of time. The same applies to the case in FIG. 8 and the like.

Here, as shown in the chest displacement image 212rd and the abdomen displacement image 222rd, when the display screen 200 of FIG. 7 is displayed, the surfaces of the chest 92 and the abdomen 94 are both located ahead the reference positions. Therefore, at this timing, in the torso displacement screen 230 illustrated in FIG. 7, the entire torso position dot row 234rd is located above the torso reference position dot row 232bk. Therefore, in the display screen 200, the entire torso position dot row 234rd is drawn in red. In this way, since the entire torso position dot row 234rd is drawn in red, the torso displacement screen 230 is displayed so that it can be easily recognized visually that the chest displacement and the abdomen displacement are synchronized with each other. Therefore, the test subject 90 who sees the display screen 200 can easily recognize that the movement of the chest 92 and the movement of the abdomen 94 are synchronized with each other. Moreover, by schematically showing the movement of the chest 92 and the movement of the abdomen 94 as shown in the torso displacement screen 230, the test subject 90 can easily recognize visually how the movement of the chest 92 and the movement of the abdomen 94 are synchronized.

The above-mentioned Patent Literature 1 discloses that the phase difference between the respiration rate of the chest and the respiration rate of the abdomen of a patient with COPD (Chronic obstructive pulmonary disease), especially, the deviation of the peak of exhalation, is detected to support the diagnosis of the patient. However, in the terminal phase of forced expiration, the rate of change in the surface position of the chest and abdomen is infinitely small, and it is difficult to detect the above phase difference. In contrast, in the first example embodiment, the displacement of the surface position of the chest and the displacement of the surface position of the abdomen can be compared. Therefore, it is possible to recognize whether the movement of the chest and the movement of the abdomen are properly synchronized even in the terminal phase of forced expiration. Therefore, it is possible to support exercises that enhance motor function efficiently.

FIG. 8 shows the display screen 200 in a case where the sign of the displacement in the anteroposterior direction of the chest 92 (chest displacement) and the sign of the displacement in the anteroposterior direction of the abdomen 94 (abdomen displacement) are both negative. As in the case of FIG. 7, FIG. 8 shows the display screen 200 displaying the displacement at a certain timing during breathing training. The display screen 200 illustrated in FIG. 8 is displayed on the display device 40 at that timing.

As in the case of FIG. 7, the display screen 200 illustrated in FIG. 8 displays the chest displacement screen 210, the abdomen displacement screen 220, and the torso displacement screen 230. In FIG. 8, the chest displacement screen 210 displays a chest displacement image 212bu indicating that the sign of the chest displacement is negative. The abdomen displacement screen 220 displays an abdomen displacement image 222bu indicating that the sign of the abdomen displacement is negative. Here, the chest displacement image 212bu and the abdomen displacement image 222bu are drawn in blue, which is the same color representation. The vertical length of each of the chest displacement image 212bu and the abdomen displacement image 222bu indicates a scalar value of the displacement. That is, like the chest displacement image 212rd, the chest displacement image 212bu indicates the chest displacement at the timing when the display screen 200 (the chest displacement image 212bu) is displayed. Similarly, the abdomen displacement image 222bu indicates the abdomen displacement at the timing when the display screen 200 (the abdomen displacement image 222bu) is displayed.

Here, in the example of FIG. 8, since the sign of the chest displacement and the sign of the abdomen displacement are both negative, the chest displacement and the abdomen displacement are synchronized with each other. The chest displacement image 212bu and the abdomen displacement image 222bu are both drawn in blue. Therefore, in the example of FIG. 8, the chest displacement image 212bu and the abdomen displacement image 222bu are displayed so that it can be easily recognized visually that the chest displacement and the abdomen displacement are synchronized with each other. Therefore, the test subject 90 who sees the display screen 200 shown in FIG. 8 can easily recognize that the movement of the chest 92 and the movement of the abdomen 94 are synchronized with each other. Therefore, the test subject 90 can easily recognize his/her own breathing condition.

As in the case of FIG. 7, in FIG. 8, the torso displacement screen 230 displays a set of points (dot row) corresponding to a three-dimensional point cloud of the torso of the test subject 90 during breathing training. The torso reference position dot row 232bk is substantially the same as that shown in FIG. 7. Further, in FIG. 8, the torso position dot row 234bu is shown as a dot row composed of a plurality of dots each formed by an open triangle. As in the case of the torso position dot row 234rd, the torso position dot row 234bu indicates a torso displacement at the timing when the display screen 200 (the torso position dot row 234bu) is displayed. That is, the torso position dot row 234bu corresponds to the displacement image described above. The torso position dot row 234bu is drawn in blue when the torso position is located behind the torso reference position (that is, when the torso displacement is negative).

Here, as shown in the chest displacement image 212bu and the abdomen displacement image 222bu, when the display screen 200 of FIG. 8 is displayed, the surfaces of the chest 92 and the abdomen 94 are both located behind the reference positions. Therefore, in the torso displacement screen 230 illustrated in FIG. 8, the entire torso position dot row 234bu is located below the torso reference position dot row 232bk. Therefore, in the display screen 200, the entire body position dot row 234bu is drawn in blue. In this way, since the entire body position dot row 234bu is drawn in blue, the torso displacement screen 230 is displayed so that it can be easily recognized visually that the chest displacement and the abdomen displacement are synchronized with each other. Therefore, the test subject 90 who sees the display screen 200 can easily recognize that the movement of the chest 92 and the movement of the abdomen 94 are synchronized with each other. As in the case of FIG. 7, by schematically showing the movement of the chest 92 and the movement of the abdomen 94 as shown in the torso displacement screen 230, the test subject 90 can easily recognize visually how the movement of the chest 92 and the movement of the abdomen 94 are synchronized. Therefore, the test subject 90 can easily recognize his/her own breathing condition.

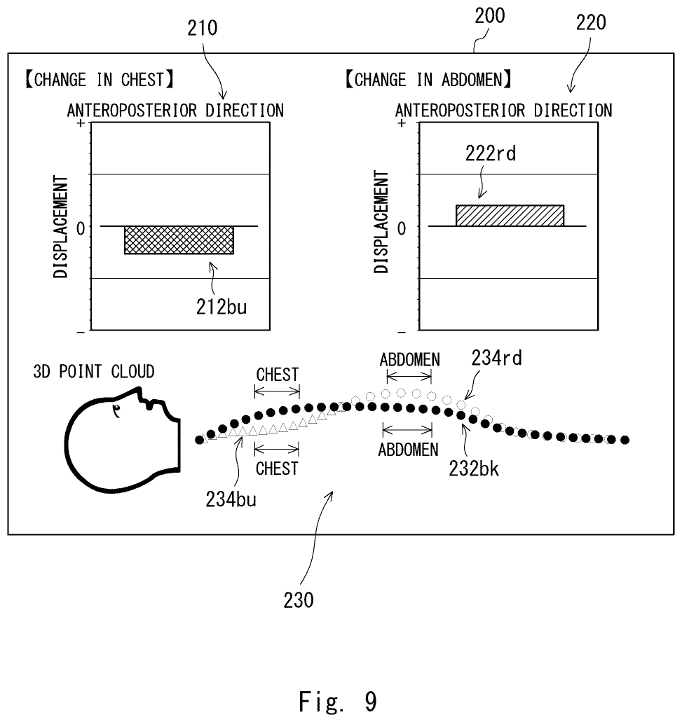

FIG. 9 shows the display screen 200 in a case where the sign of the displacement in the anteroposterior direction of the chest 92 (chest displacement) and the sign of the displacement in the anteroposterior direction of the abdomen 94 (abdomen displacement) are different from each other. FIG. 9 shows a display screen 200 in a case where the sign of the chest displacement is negative and the sign of the abdomen displacement is positive. As in the case of FIG. 7 and the like, FIG. 9 shows the display screen 200 displaying the displacement at a certain timing during breathing training. The display screen 200 illustrated in FIG. 9 is displayed on the display device 40 at that timing.

As in the case of FIG. 7 and the like, the display screen 200 illustrated in FIG. 9 displays the chest displacement screen 210, the abdomen displacement screen 220, and the torso displacement screen 230. In FIG. 9, the chest displacement screen 210 displays in blue a chest displacement image 212bu indicating that the sign of the chest displacement is negative. The abdomen displacement screen 220 displays in red an abdomen displacement image 222rd indicating that the sign of the abdomen displacement is positive. Therefore, the chest displacement image 212bu and the abdomen displacement image 222rd are drawn in different color representations from each other.

Here, in the example of FIG. 9, since the sign of the chest displacement is negative and the sign of the abdomen displacement is positive, the chest displacement and the abdomen displacement are not synchronized with each other. The chest displacement image 212bu is drawn in blue, and the abdomen displacement image 222rd is drawn in red. That is, the chest displacement image 212bu and the abdomen displacement image 222rd are drawn in different color representations from each other. Therefore, in the example of FIG. 9, the chest displacement image 212bu and the abdomen displacement image 222rd are displayed so that it can be easily recognized visually that the chest displacement and the abdomen displacement are not synchronized with each other. Therefore, the test subject 90 who sees the display screen 200 shown in FIG. 9 can easily recognize that the movement of the chest 92 and the movement of the abdomen 94 are not synchronized with each other.

As in the case of FIG. 7, in FIG. 9, the torso displacement screen 230 displays a set of points (dot row) corresponding to a three-dimensional point cloud of the torso of the test subject 90 during breathing training. The torso reference position dot row 232bk is substantially the same as that shown in FIG. 7. In FIG. 9, the torso displacement screen 230 displays the torso position dot row 234rd drawn in red and the torso position dot row 234bu drawn in blue.

As shown in the chest displacement image 212bu and the abdomen displacement image 222rd, when the display screen 200 in FIG. 9 is displayed, the surface of the chest 92 is located behind the reference position, and the surface of the abdomen 94 is located ahead the reference position. Therefore, in the torso displacement screen 230 illustrated in FIG. 9, the torso position dot line 234bu drawn in blue is shown below the torso reference position dot line 232bk at a position corresponding to the chest 92. On the other hand, the torso position dot line 234rd drawn in red is shown above the torso reference position dot line 232bk at a position corresponding to the abdomen 94. As described above, in the torso displacement screen 230 illustrated in FIG. 9, at the torso portion located ahead the reference position, the torso position dot line 234rd is shown above the torso reference position dot line 232bk. On the other hand, at the torso portion located behind the reference position, the torso position dot row 234bu is shown below the torso reference position dot row 232bk.

In this way, since the torso position dot rows 234rd and 234bu are displayed in different color representations from each other, the torso displacement screen 230 is displayed so that it can be easily recognized visually that the chest displacement and the abdomen displacement are not synchronized with each other. Therefore, the test subject 90 who sees the display screen 200 can easily recognize that the movement of the chest 92 and the movement of the abdomen 94 are not synchronized with each other. As in the case of FIG. 7, by schematically showing the movement of the chest 92 and the movement of the abdomen 94 as shown in the torso displacement screen 230, the test subject 90 can easily recognize visually how the movement of the chest 92 and the movement of the abdomen 94 are not synchronized. Therefore, the test subject 90 can easily recognize his/her own breathing condition.

The display screen 200 shown in FIGS. 7 to 9 displays the displacement at the timing when the display screen 200 is displayed. Therefore, the display screen 200 displays the displacement in real time during the breathing training of the test subject 90. Therefore, the test subject 90 who sees the display screen 200 can immediately recognize whether or not the breathing method at the current timing is correct during his/her breathing training. That is, when the test subject 90 sees the display screen 200 shown in FIGS. 7 and 8, he/she can immediately recognize that the breathing method at the current timing is correct during his/her breathing training. On the other hand, when the test subject 90 sees the display screen 200 shown in FIG. 9, he/she can immediately recognize that the breathing method at the current timing is incorrect during his/her breathing training. Therefore, the test subject 90 can easily recognize his/her breathing condition in real time.

In the display screen 200, the display of each of the chest displacement image 212, the abdomen displacement image 222 and the torso position dot row 234 changes with the passage of time of the breathing training. Therefore, the test subject 90 who sees the display screen 200 can easily visually recognize the movement of his/her chest 92 and abdomen 94 (and torso) with the passage of time of the breathing training.

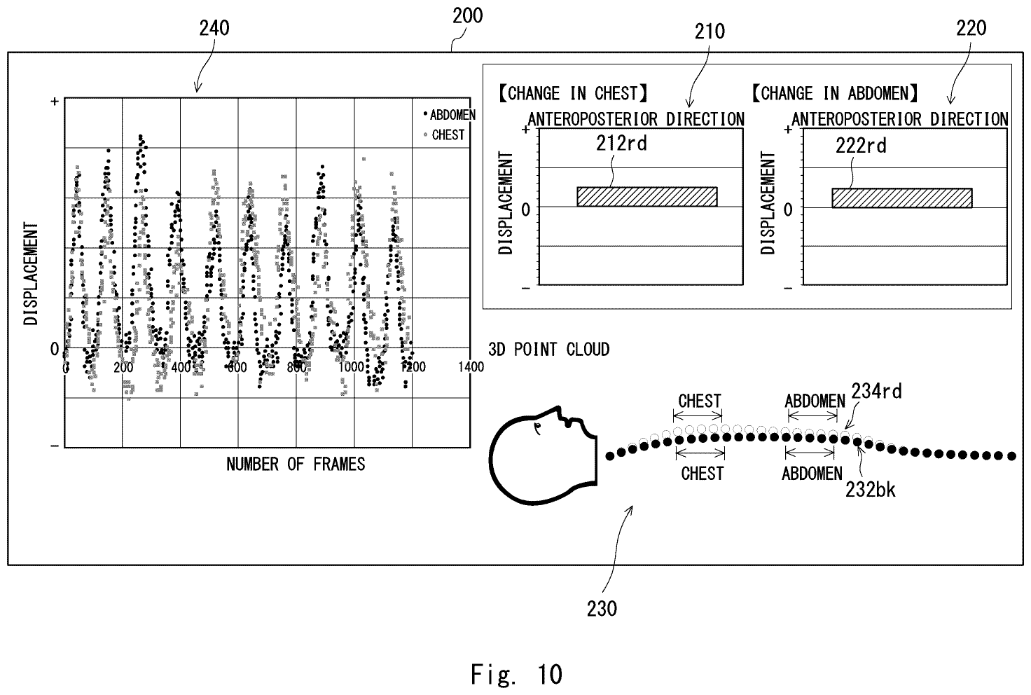

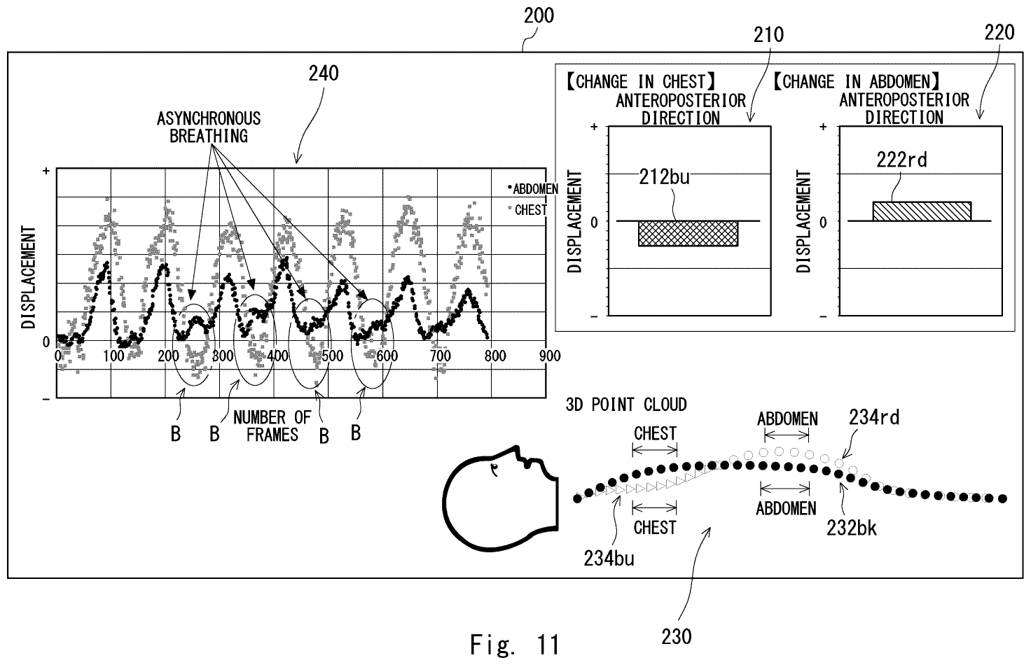

FIGS. 10 and 11 show examples of the display screen 200 on which the displacement history screen 240 for displaying the history of the displacement is displayed. FIG. 10 shows an example of the case where the displacement history screen 240 is displayed on the display screen 200 shown in FIG. 7. FIG. 11 shows an example of the case where the displacement history screen 240 is displayed on the display screen 200 shown in FIG. 9. The displacement history screen 240 may be displayed separately from the chest displacement screen 210, the abdomen displacement screen 220, and the torso displacement screen 230, shown in FIG. 7 and the like.

The displacement history screen 240 displays a graph in which the vertical axis indicates the displacement of each of the chest 92 and abdomen 94, and the horizontal axis indicates the transition of the number of frames of the photographed moving image. That is, in the graph shown in the displacement history screen 240, the horizontal axis indicates the passage of time. Therefore, the displacement history screen 240 indicates time series data of the chest displacement and the abdomen displacement. For example, when the frame rate of the photographing device 30 is 15 fps (frames per second), the data at the number of frames “600” on the horizontal axis corresponds to the data at 40 seconds after the start of photographing. In the displacement history screen 240, the points corresponding to the chest 92 and the points corresponding to the abdomen 94 indicate the displacement of the chest 92 (chest displacement) and the displacement of the abdomen 94 (abdomen displacement), respectively, at the timing when the corresponding number of frames is obtained.

The displacement history screen 240 may display an image of the history of the displacement that changes with the passage of time, during breathing training. Therefore, the display of the displacement history screen 240 changes with the passage of time. In FIGS. 10 and 11, the chest displacement and the abdomen displacement are shown in the same graph, but may be shown in separate graphs.

In the displacement history screen 240 shown in FIG. 10, the phase of the graph shape (waveform) indicating the change of the chest displacement and the phase of the graph shape (waveform) indicating the change of the abdomen displacement generally coincide with each other. That is, the abdomen displacement decreases at approximately the same timing as the chest displacement decreases, and the abdomen displacement increases at approximately the same timing as the chest displacement increases. Therefore, according to the displacement history screen 240 shown in FIG. 10, it can be recognized that the movement of the chest 92 (displacement) and the movement of the abdomen 94 (displacement) are synchronized with each other. Therefore, the test subject 90 can easily recognize his/her own breathing condition.

On the other hand, in the displacement history screen 240 shown in FIG. 11, the phase of the graph shape (waveform) indicating the change in the chest displacement does not coincide with the phase of the graph shape (waveform) indicating the change in the abdomen displacement. Specifically, at the point indicated by arrows B, the abdomen displacement increases at the timing when the chest displacement decreases. Therefore, according to the displacement history screen 240 shown in FIG. 11, it can be recognized that asynchronous breathing occurs, i.e., the movement of the chest 92 and the movement of the abdomen 94 are not synchronized with each other at the point indicated by the arrows B. Therefore, the test subject 90 can easily recognize his/her own breathing condition.

Return to the description of the flowchart shown in FIG. 5. The movement direction detection unit 118 detects the movement direction of each of the chest 92 and the abdomen 94 (Step S120). Here, the “movement direction” is the direction in which the surface of each of the chest 92 and the abdomen 94 is moving. In other words, the movement direction indicates whether the surface of each of the chest 92 and the abdomen 94 is moving in the anterior or posterior direction. In other words, the movement direction is the direction of change (increase or decrease) of the displacement. That is, if the displacement is increasing, that is, if the test subject 90 is taking inspiration, the movement direction is anterior direction (positive direction). On the other hand, if the displacement is decreasing, that is, if the test subject 90 is taking expiration, the movement direction is posterior direction (negative direction). In other words, the movement direction can also be said to be the direction of the change rate of the displacement.

Specifically, the movement direction detection unit 118 subtracts, from the current displacement of the chest 92, the displacement of the chest 92 at a predetermined time before. When the value obtained by the subtraction is 0 or more, the movement direction detection unit 118 determines that the movement direction of the chest 92 is in the positive direction (anterior direction). On the other hand, when the value obtained by the subtraction is less than 0, the movement direction detection unit 118 determines that the movement direction of the chest 92 is in the negative direction (posterior direction).

Similarly, the movement direction detection unit 118 subtracts, from the current displacement of the abdomen 94, the displacement of the abdomen 94 at a predetermined time before. When the value obtained by the subtraction is 0 or more, the movement direction detection unit 118 determines that the movement direction of the abdomen 94 is in the positive direction (anterior direction). On the other hand, when the value obtained by the subtraction is less than 0, the movement direction detection unit 118 determines that the movement direction of the abdomen 94 is in the negative direction (posterior direction).

The “predetermined time” may be, for example, 1 second. In this case, the movement direction may correspond to the direction of the change rate of the displacement. Alternatively, the “predetermined time” may be the inverse of the frame rate. In this case, the movement direction for the frame to be processed corresponds to the direction of change from the displacement detected for the frame one step before the frame to be processed. Therefore, in this case, the movement direction detection unit 118 detects the movement direction by subtracting the displacement for the frame one step before the frame to be processed from the displacement for the frame to be processed.

The display control unit 130 performs control for displaying the direction image (Step S122). The display control unit 130 performs control for displaying the direction image, which is the movement image described above and is an image indicating the movement direction in the anteroposterior direction. The display control unit 130 performs control for displaying the chest direction image and the abdomen direction image. Here, the “chest direction image” is the chest movement image described above, and is an image (direction image) indicating the movement direction (chest movement direction) of the chest 92. That is, the chest direction image is an image indicating the direction of change (increase or decrease) of the displacement of the chest 92. The “abdomen direction image” is the abdomen movement image described above, and is an image (direction image) indicating the movement direction (abdomen movement direction) of the abdomen 94. That is, the abdomen direction image is an image indicating the direction of change (increase or decrease) of the displacement of the abdomen 94.

Here, when the chest movement direction and the abdomen movement direction are synchronized with each other, the display control unit 130 performs control so that the display form of the chest direction image and the display form of the abdomen direction image are the same as each other. On the other hand, when the chest movement direction and the abdomen movement direction are not synchronized with each other, the display control unit 130 performs control so that the display form of the chest direction image and the display form of the abdomen direction image are different from each other.

Here, the case where “the chest movement direction and the abdomen movement direction are synchronized with each other” is a case where the chest movement direction (positive direction or negative direction) and the abdomen movement direction (positive direction or negative direction) are the same as each other. Conversely, the case where “the chest movement direction and the abdomen movement direction are not synchronized with each other” is a case where the chest movement direction (positive direction or negative direction) and the abdomen movement direction (positive direction or negative direction) are different from each other. Therefore, when the chest movement direction and the abdomen movement direction are the same as each other, the display control unit 130 performs control so that the display form of the chest direction image and the display form of the abdomen direction image are the same as each other. On the other hand, when the chest movement direction and the abdomen movement direction are different from each other, the display control unit 130 performs control so that the display form of the chest direction image and the display form of the abdomen direction image are different from each other.

The “display form” is, for example, a color representation. In this case, when the chest movement direction and the abdomen movement direction are synchronized with each other, the display control unit 130 performs control so that the color representation of the chest direction image and the color representation of the abdomen direction image are the same as each other. That is, when the chest movement direction and the abdomen movement direction are the same as each other, the display control unit 130 performs control so that the color representation of the chest direction image and the color representation of the abdomen direction image are the same as each other. On the other hand, when the chest movement direction and the abdomen movement direction are different from each other, the display control unit 130 performs control so that the color representation of the chest direction image and the color representation of the abdomen direction image are different from each other. Note that the display form is not limited to the color representation. In the first example embodiment, the “display form” is assumed to be the color representation.

For example, for each of the chest direction image and the abdomen direction image, the display control unit 130 performs control so that the movement direction is displayed in red when the movement direction is “positive direction (anterior direction)”. Therefore, when the chest movement direction is “positive direction (anterior direction)”, the chest direction image is displayed in red. Similarly, when the abdominal movement direction is “positive direction (anterior direction)”, the abdomen direction image is displayed in red. On the other hand, for each of the chest direction image and the abdomen direction image, the display control unit 130 performs control so that the movement direction is displayed in blue when the movement direction is “negative direction (posterior direction)”. Therefore, when the chest direction is “negative direction (posterior direction)”, the chest direction image is displayed in blue. Similarly, when the abdomen direction is “negative direction (posterior direction)”, the abdomen direction image is displayed in blue.

The display control unit 130 generates data indicating an instruction for displaying the direction image (direction image display instruction data) and transmits the data to the display device 40. The direction image display instruction data may indicate the movement direction and a color representation to be displayed according to the movement direction. In particular, the direction image display instruction data may indicate the chest movement direction and a color representation to be displayed according to the chest movement direction, and the abdomen movement direction and a color representation to be displayed according to the abdomen movement direction. The direction image display instruction data may indicate the torso movement direction and a color representation to be displayed according to the torso movement direction. Thus, the display device 40 displays the direction image in a color representation according to the movement direction.

The display control unit 130 performs control so that the direction image is displayed so as to change in real time in accordance with the progress of the breathing training. That is, the display control unit 130 causes the display device 40 to display the direction image that changes in accordance with the progress of the breathing training of the test subject 90. In other words, the display control unit 130 performs control so that the direction image (movement image) that changes in accordance with the progress of the breathing training of the test subject 90 is displayed during the progress of the breathing training. In other words, the display control unit 130 performs control so that the direction image corresponding to the change of the displacement at a certain timing during the breathing training is displayed at that timing.

The display control unit 130 generates direction image display instruction data every time each frame of the photographed image of the test subject 90 performing the breathing training is acquired, and transmits the data to the display device 40. Accordingly, the display control unit 130 can cause the display device 40 to display the direction image that changes with the progress of the breathing training.

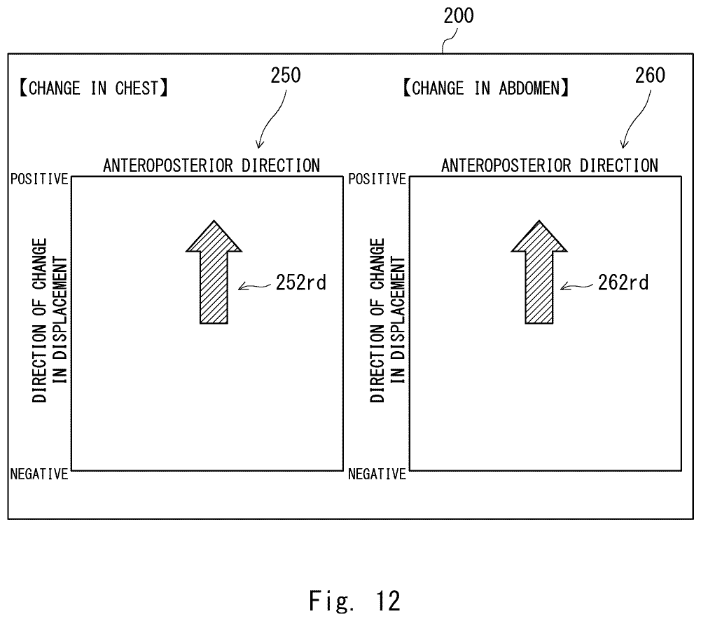

FIGS. 12 to 14 show examples of display screens 200 that are displayed on the display device 40 under the control of the display control unit 130 according to the first example embodiment and indicate the movement directions in the anteroposterior direction of the chest 92 and the abdomen 94. FIG. 12 shows the display screen 200 in a case where the movement direction in anteroposterior direction of the chest 92 (chest movement direction) and the movement direction in anteroposterior direction of the abdomen 94 (abdomen movement direction) are both in the positive direction (anterior direction).

FIG. 12 shows the display screen 200 displaying the movement direction of (the direction of change of the displacement) at a certain timing during breathing training. The display screen 200 shown in FIG. 12 is displayed on the display device 40 at that timing. That is, the display screen 200 shown in FIG. 12 is displayed on the display device 40 in real time during breathing training.

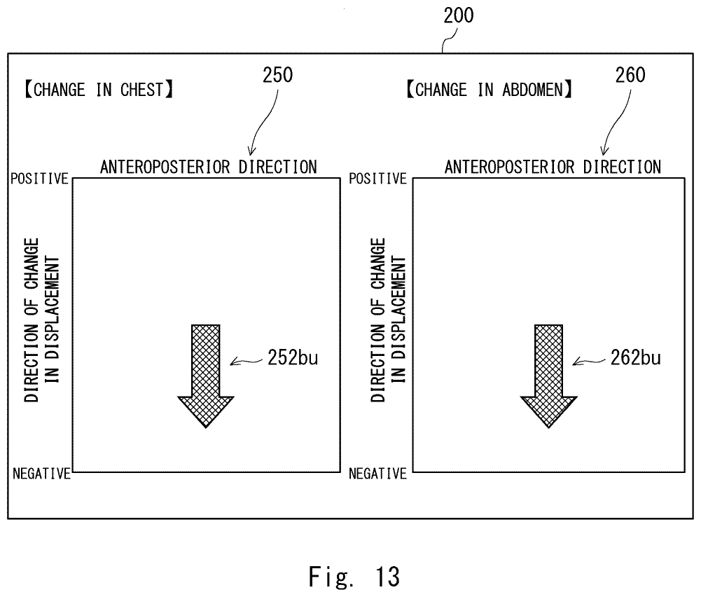

The display screen 200 shown in FIG. 12 displays the chest direction screen 250 and the abdomen direction screen 260. The display screen 200 may display the chest direction screen 250 and the abdomen direction screen 260 together with the chest displacement screen 210, the abdomen displacement screen 220 and the torso displacement screen 230 shown in FIG. 7 and the like. The same applies to the case in FIG. 13 and the like.

In FIG. 12, the chest direction screen 250 displays a chest direction image 252rd indicating that the chest movement direction is in the positive direction. The abdomen direction screen 260 displays an abdomen direction image 262rd indicating that the abdomen movement direction is in the positive direction. Here, the chest direction image 252rd and the abdomen direction image 262rd are drawn in red, which is the same color representation. That is, the chest direction image 252rd indicates the chest movement direction at the timing when the display screen 200 (chest direction image 252rd) is displayed. Similarly, the abdomen direction image 262rd indicates the abdomen movement direction at the timing when the display screen 200 (abdomen direction image 262rd) is displayed. The vertical length of the chest direction image 252 and the abdomen direction image 262 may indicate a difference in the displacement, that is, a scalar value of the change rate of the displacement. The same applies to the case in FIG. 13 and the like.

Here, the chest movement direction and the abdomen movement direction change with the passage of time. Therefore, in the display screen 200, the chest direction image 252 and the abdomen direction image 262 change with the passage of time. The same applies to the case in FIG. 13 and the like.

Here, in the example of FIG. 12, since both the chest movement direction and the abdomen movement direction are in the positive direction (anterior direction), the chest movement direction and the abdomen movement direction are synchronized with each other. The chest direction image 252rd and the abdomen direction image 262rd are both drawn in red. Therefore, in the example of FIG. 12, the chest direction image 252rd and the abdomen direction image 262rd are displayed so that it can be easily recognized visually that the chest movement direction and the abdomen movement direction are synchronized with each other. Therefore, the test subject 90 who sees the display screen 200 shown in FIG. 12 can easily recognize that the movement of the chest 92 and the movement of the abdomen 94 are synchronized with each other. Therefore, the test subject 90 can easily recognize his/her own breathing condition.

FIG. 13 shows the display screen 200 in a case where the movement direction in anteroposterior direction of the chest 92 (chest movement direction) and the movement direction in anteroposterior direction of the abdomen 94 (abdomen movement direction) are both in the negative direction (posterior direction). As in the case of FIG. 12, FIG. 13 shows the display screen 200 displaying the movement direction (the direction of change of the displacement) at a certain timing during breathing training. The display screen 200 shown in FIG. 13 is displayed on the display device 40 at that timing.

As in the case of FIG. 12, the display screen 200 shown in FIG. 13 displays the chest direction screen 250 and the abdomen direction screen 260. In FIG. 13, the chest direction screen 250 displays a chest direction image 252bu indicating that the chest movement direction is in the negative direction. The abdomen direction screen 260 displays an abdomen direction image 262bu indicating that the abdomen movement direction is in the negative direction. Here, the chest direction image 252bu and the abdomen direction image 262bu are drawn in blue, which is the same color representation. That is, the chest direction image 252bu indicates the chest movement direction at the timing when the display screen 200 (chest direction image 252bu) is displayed. Similarly, the abdomen direction image 262bu indicates the abdomen movement direction at the timing when the display screen 200 (abdomen direction image 262bu) is displayed.

Here, in the example of FIG. 13, since both the chest movement direction and the abdomen movement direction are in the negative direction (posterior direction), the chest movement direction and the abdomen movement direction are synchronized with each other. The chest direction image 252bu and the abdomen direction image 262bu are both drawn in blue. Therefore, in the example of FIG. 13, the chest direction image 252bu and the abdomen direction image 262bu are displayed so that it can be easily recognized visually that the chest movement direction and the abdomen movement direction are synchronized with each other. Therefore, the test subject 90 who sees the display screen 200 shown in FIG. 13 can easily recognize that the movement of the chest 92 and the movement of the abdomen 94 are synchronized with each other. Therefore, the test subject 90 can easily recognize his/her own breathing condition.

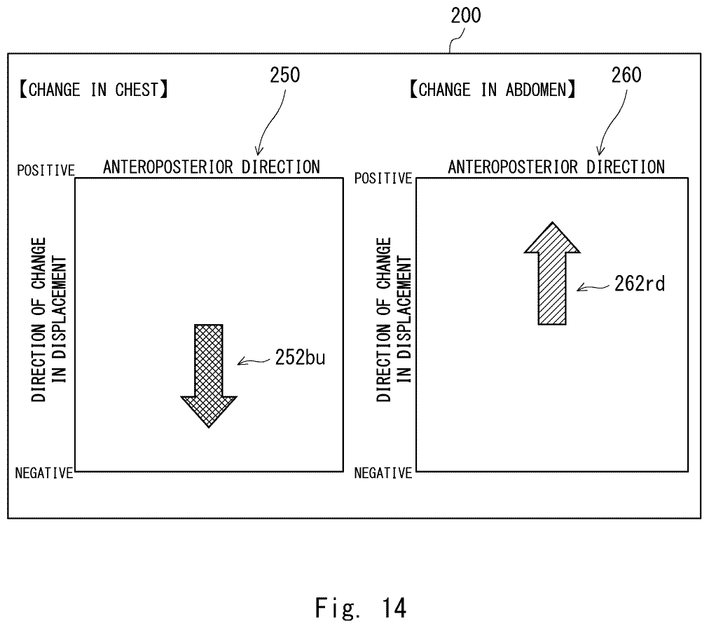

FIG. 14 shows the display screen 200 in a case where the movement direction in anteroposterior direction of the chest 92 (chest movement direction) and the movement direction in anteroposterior direction of the abdomen 94 (abdomen movement direction) are different from each other. FIG. 14 shows the display screen 200 in a case where the chest movement direction is in the negative direction (posterior direction) and the abdomen movement direction is in the positive direction (anterior direction). As in the case of FIG. 12 and the like, FIG. 14 shows the display screen 200 displaying the movement direction (the direction of change of the displacement) at a certain timing during breathing training. The display screen 200 shown in FIG. 14 is displayed on the display device 40 at that timing.

As in the case of FIG. 12 and the like, the display screen 200 shown in FIG. 14 displays the chest direction screen 250 and the abdomen direction screen 260. In FIG. 14, the chest direction screen 250 displays in blue a chest direction image 252bu indicating that the chest movement direction is in the negative direction. The abdomen direction screen 260 displays in red an abdomen direction image 262rd indicating that the abdomen movement direction is in the positive direction. Here, the chest direction image 252bu and the abdomen direction image 262rd are drawn in different color representations.

Here, in the example of FIG. 14, since the chest movement direction is in the negative direction (posterior direction) and the abdomen movement direction is in the positive direction (anterior direction), the chest movement direction and the abdomen movement direction are not synchronized with each other. The chest direction image 252bu is drawn in blue, and the abdomen direction image 262rd is drawn in red. Therefore, in the example of FIG. 14, the chest direction image 252bu and the abdomen direction image 262rd are displayed so that it can be easily recognized visually that the chest movement direction and the abdomen movement direction are not synchronized with each other. Therefore, the test subject 90 who sees the display screen 200 shown in FIG. 14 can easily recognize that the movement of the chest 92 and the movement of the abdomen 94 are not synchronized with each other. Therefore, the test subject 90 can easily recognize his/her own breathing condition.

The display screen 200 shown in FIGS. 12 to 14 displays the movement direction (direction of change of displacement) at the timing when the display screen 200 is displayed. Therefore, the display screen 200 displays the movement direction in real time during the breathing training of the test subject 90. Therefore, the test subject 90 who sees the display screen 200 can immediately recognize whether or not the breathing method at the current timing is correct during his/her breathing training. That is, when the test subject 90 sees the display screen 200 shown in FIGS. 12 and 13, he/she can immediately recognize that the breathing method at the current timing is correct during his/her breathing training. On the other hand, when the test subject 90 sees the display screen 200 shown in FIG. 14, he/she can immediately recognize that the breathing method at the current timing is incorrect during his/her breathing training. Therefore, the test subject 90 can easily recognize his/her breathing condition in real time.

Return to the description of the flowchart shown in FIG. 5. The width detection unit 120 detects changes in the width of each of the chest 92 and the abdomen 94 (Step S130). That is, the width detection unit 120 detects the displacement in the lateral direction of each of the chest 92 and the abdomen 94. Specifically, the width detection unit 120 detects the positions in the lateral direction (width direction) of each of the chest 92 and the abdomen 94. More specifically, the width detection unit 120 specifies the chest region and the abdomen region in the photographed image 52 of the test subject 90 acquired in the process of S106. This specifying process may be performed by using the process of specifying the regions corresponding to the chest 92 and the abdomen 94, respectively, in S104.

In addition, the width detection unit 120 detects the positions of the chest 92 and the abdomen 94, respectively, in the lateral directions in the same manner as when the reference positions of the chest 92 and the abdomen 94, respectively, in the lateral direction are specified in the process of S104. Specifically, the width detection unit 120 detects the width of the chest 92 (chest width) by using the position information corresponding to the chest region specified in the photographed image 52 acquired in the process of S106. Similarly, the width detection unit 120 detects the width of the abdomen 94 (abdomen width) by using the position information corresponding to the abdomen region specified in the photographed image 52 acquired in the process of S106.

Furthermore, the width detection unit 120 calculates the displacement of the chest width relative to the chest reference width specified in S104. The width detection unit 120 calculates a difference between the chest width and the chest reference width as the displacement in the lateral direction (chest width increase/decrease). Here, when the chest width is larger (wider) than the chest reference width, the sign of the displacement in the lateral direction is positive (+). On the other hand, when the chest width is smaller (narrower) than the chest reference width, the sign of the displacement in the lateral direction is negative (−). Therefore, the width detection unit 120 calculates the displacement in the lateral direction of the chest 92 by subtracting the value of the chest reference width from the value of the chest width. The chest width may be, for example, the distance between the left and right ends of the chest 92.

Furthermore, the width detection unit 120 calculates the displacement of the abdomen width relative to the abdomen reference width specified in S104. The width detection unit 120 calculates the difference between the abdomen width and the abdomen reference width as the displacement in the lateral direction (abdomen width increase/decrease). Here, when the abdomen width is larger (wider) than the abdomen reference width, the sign of the displacement in the lateral direction is positive (+). On the other hand, when the abdomen width is smaller (narrower) than the abdomen reference width, the sign of the displacement in the lateral direction is negative (−). Therefore, the width detection unit 120 calculates the displacement in the lateral direction of the abdomen 94 by subtracting the value of the abdomen reference width from the value of the abdomen width. Similarly, the abdomen width may be, for example, the distance between the left and right ends of the abdomen 94. Note that the width detection unit 120 does not need to calculate the displacement in the lateral direction of the abdomen 94. That is, the width detection unit 120 may calculate the displacement in the lateral direction of at least chest 92.

The display control unit 130 performs control for displaying the width image (Step S132). The width image is the movement image described above and is an image indicating an increase/decrease or change of the width from the reference width (displacement in the lateral direction). That is, the display control unit 130 performs control so that the displacement in the lateral direction of the chest 92 and abdomen 94 of the test subject 90 is displayed. The display control unit 130 performs control for displaying a chest width image and an abdomen width image. The “chest width image” is the chest movement image described above, and is an image (width image) indicating change (increase or decrease) in the width of the chest 92 from the chest reference width. That is, the chest width image is an image indicating the displacement of the chest width relative to the chest reference width (chest width increase/decrease). Further, the “abdomen width image” is the abdomen movement image described above, and is an image (width image) indicating change (increase/decrease) of the width of the abdomen 94 from the abdomen reference width. That is, the abdomen width image is an image indicating the displacement of the abdomen width relative to the abdomen reference width (the abdomen width increase/decrease).

Here, the display control unit 130 may perform control so that the display form of the chest width image changes when the increase/decrease of the chest width falls below the threshold Th1 (<0) and when it does not. For example, the display control unit 130 may perform control so that the chest width image is displayed in blue when the increase/decrease of the chest width does not fall below the threshold Th1, and the chest width image is displayed in red when the increase/decrease of the chest width falls below the threshold Th1.

The display control unit 130 generates data indicating an instruction for displaying the width image (width image display instruction data) and transmits the data to the display device 40. The width image display instruction data may indicate a width increase/decrease. In particular, the width image display instruction data may indicate a chest width increase/decrease and an abdomen width increase/decrease. Further, the width image display instruction data may indicate a color representation of the chest width image drawn in response to the comparison between the chest width increase/decrease and the threshold Th1 (<0).

The display control unit 130 performs control so that the width image so as to change in real time in accordance with the progress of the breathing training. That is, the display control unit 130 causes the display device 40 to display the width image that changes in accordance with the progress of the breathing training of the test subject 90. In other words, the display control unit 130 performs control so that the width image (movement image) that changes in accordance with the progress of the breathing training of the test subject 90 is displayed during the progress of the breathing training. In other words, the display control unit 130 performs control so that the width image corresponding to the width increase/decrease at a certain timing during the breathing training is displayed at that timing.

The display control unit 130 generates the width image display instruction data every time each frame of the photographed image of the test subject 90 performing breathing training is acquired, and transmits the data to the display device 40. Accordingly, the display control unit 130 can cause the display device 40 to display the width image that changes with the progress of the breathing training.

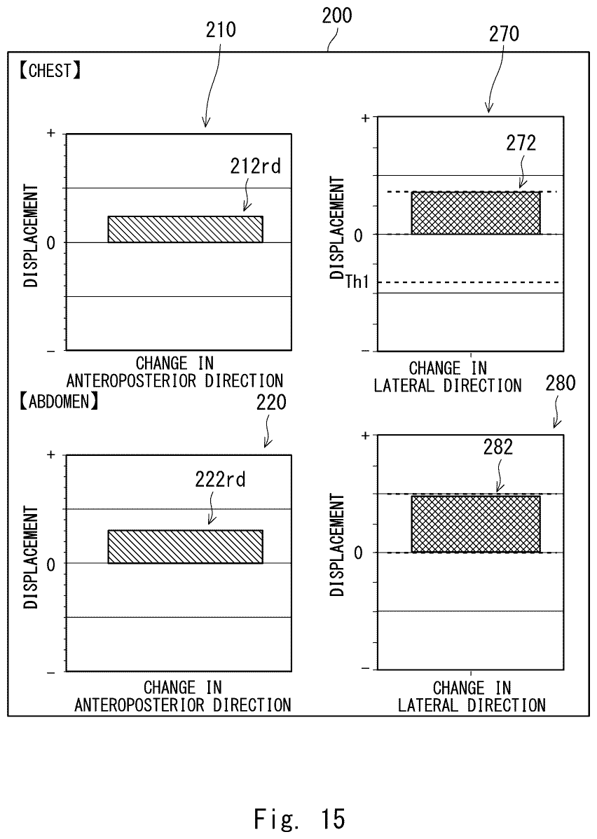

FIG. 15 shows an example of a display screen 200 that is displayed on the display device 40 under the control of the display control unit 130 according to the first example embodiment and indicates the displacements in the lateral direction of the chest 92 and the abdomen 94. The display screen 200 shown in FIG. 15 displays the chest width screen 270 and the abdomen width screen 280 together with the chest displacement screen 210 and the abdomen displacement screen 220 shown in FIG. 7. The display screen 200 may display the chest width screen 270 and the abdomen width screen 280 separately from the chest displacement screen 210 and the abdomen displacement screen 220.

The chest width screen 270 displays in blue a chest width image 272 indicating the chest width increase/decrease (the displacement in the lateral direction of the chest 92). The abdomen width screen 280 displays in blue an abdomen width image 282 indicating the abdomen width increase/decrease (the displacement in the lateral direction of the abdomen 94). The chest width screen 270 displays the threshold Th1. When the chest width increase/decrease is below the threshold Th1 on the chest width screen 270, the chest width image 272 may turn to red, for example. The color representation of the chest width image 272 may change according to the difference between the chest width increase/decrease and the threshold Th1. For example, the color representation may gradually turn from blue to red as the chest width increase/decrease (chest width image 272) approaches the threshold Th1 during expiration.

As described above, under control by the display control unit 130, the display device 40 displays the chest width image 272 indicating the displacement of the chest 92 in the lateral direction. Accordingly, the test subject 90 can easily visually recognize the movement of the chest 92 in the lateral direction during breathing training by viewing the chest width image 272 (chest width screen 270). Furthermore, the chest width screen 270 displays the threshold Th1. Therefore, if the chest width image 272 shows that the chest width is below the threshold Th1 at the time of expiration, the test subject 90 can easily visually recognize that the rib internal-rotation is sufficiently performed. On the other hand, if the chest width image 272 does not show that the chest width is below the threshold Th1 at the time of expiration, the test subject 90 can easily visually recognize that the rib internal-rotation is not sufficiently performed. Thus, the test subject 90 can easily recognize his/her own breathing condition.

Further, the display screen 200 shown in FIG. 15 displays the displacement (chest width) in the lateral direction of the chest 92 at the timing when the display screen 200 is displayed. Accordingly, the display screen 200 displays the chest width in real time during the breathing training of the test subject 90. Therefore, the test subject 90 who sees the display screen 200 can immediately recognize whether or not the breathing method at the current timing is correct during his/her breathing training. Therefore, the test subject 90 can easily recognize his/her own breathing condition in real time.

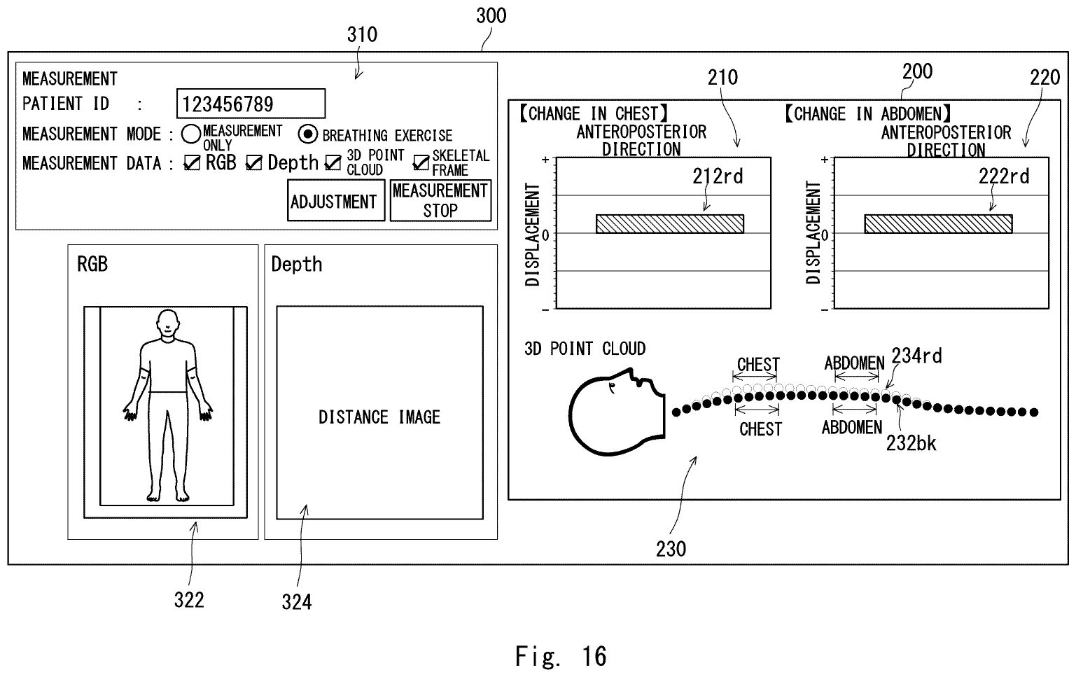

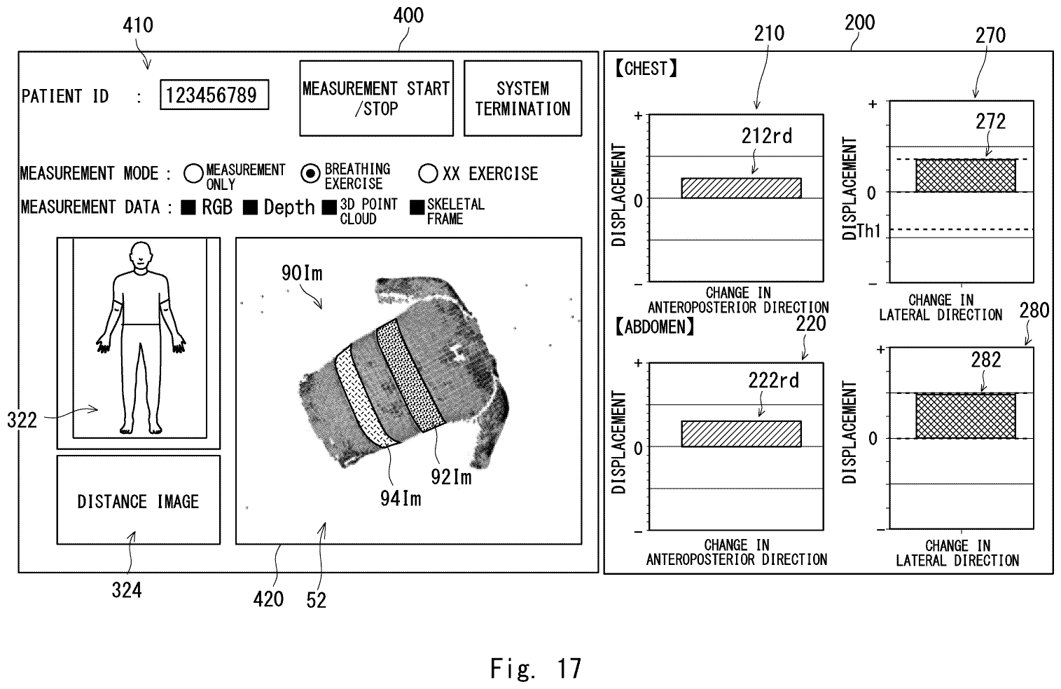

FIGS. 16 and 17 show examples of instructor-aimed display screens according to the first example embodiment. The instructor-aimed display screen may be displayed on the display device 40. The instructor-aimed display screen may be displayed on a display device carried by the instructor, which is different from the display device 40 visible to the test subject 90. The instructor-aimed display screen may be displayed on the interface 108 of the information processing apparatus 100.

FIG. 16 shows an example of an instructor-aimed display screen 300, which is a first example of the instructor-aimed display screen. The instructor-aimed display screen 300 displays the chest displacement screen 210, the abdomen displacement screen 220, and the torso displacement screen 230 shown in FIG. 7 and the like. Furthermore, the instructor-aimed display screen 300 displays a setting screen 310, an RGB image display screen 322, and a distance image display screen 324. The setting screen 310 is used for setting for breathing training. The RGB image display screen 322 displays an RGB image obtained by the photographing device 30 photographing the test subject 90. The distance image display screen 324 displays a distance image obtained by the photographing device 30 photographing the test subject 90. The instructor can confirm whether the test subject 90 is properly photographed by the photographing device 30, by visually inspecting the RGB image display screen 322 and the distance image display screen 324.

FIG. 17 shows an example of an instructor-aimed display screen 400, which is a second example of the instructor-aimed display screen. The instructor-aimed display screen 400 displays the chest displacement screen 210, the abdomen displacement screen 220, the chest width screen 270, and the abdomen width screen 280 shown in FIG. 15. Further, the instructor-aimed display screen 300 displays the setting screen 410, the RGB image display screen 322, the distance image display screen 324, and the photographed image display screen 420. The setting screen 410 is used for setting for breathing training. The photographed image display screen 420 displays the photographed image 52.

Thus, by displaying the instructor-aimed display screens 300 and 400 separately from the display screen 200 visible to the test subject 90, it is possible to suppress the test subject 90 from seeing information that is not necessary for the test subject 90. Therefore, the test subject 90 can perform breathing training intensively.

Return to the description of the flowchart shown in FIG. 5. The result determination unit 140 determines the evaluation of the result of breathing training (Step S140). The result determination unit 140 generates evaluation information related to the evaluation of the training result.

For example, the result determination unit 140 determines whether or not the movement of the chest 92 is synchronized with the movement of the abdomen 94. The result determination unit 140 generates evaluation information indicating whether or not the movement of the chest 92 is synchronized with the movement of the abdomen 94.

Specifically, the result determination unit 140 determines whether or not the sign of the chest displacement and the sign of the abdomen displacement are the same as each other. When the sign of the chest displacement and the sign of the abdomen displacement are the same as each other, the result determination unit 140 may determine that the movement of the chest 92 and the movement of the abdomen 94 are synchronized with each other. On the other hand, when the sign of the chest displacement and the sign of the abdomen displacement are different from each other, the result determination unit 140 may determine that the movement of the chest 92 and the movement of the abdomen 94 are not synchronized with each other.

The result determination unit 140 determines whether or not the chest movement direction and the abdomen movement direction are the same as each other. When the chest movement direction and the abdomen movement direction are the same as each other, the result determination unit 140 may determine that the movement of the chest 92 and the movement of the abdomen 94 are synchronized with each other. On the other hand, when the chest movement direction and the abdomen movement direction are different from each other, the result determination unit 140 may determine that the movement of the chest 92 and the movement of the abdomen 94 are not synchronized with each other. Alternatively, when the sign of the chest displacement and the sign of the abdomen displacement are the same as each other and the chest movement direction and the abdomen movement direction are the same as each other, the result determination unit 140 may determine that the movement of the chest 92 and the movement of the abdomen 94 are synchronized with each other. On the other hand, when the sign of the chest displacement and the sign of the abdomen displacement are different from each other, or when the chest movement direction and the abdomen movement direction are different from each other, the result determining unit 140 may determine that the movement of the chest 92 and the movement of the abdomen 94 are not synchronized with each other.

Alternatively, the result determining unit 140 may determine whether or not the movement of the chest 92 and the movement of the abdomen 94 are synchronized with each other by using a learned model learned by machine learning, for example, a neural network. The learned model is learned by supervised learning, for example. For example, the learned model is learned so that the chest displacement and the abdomen displacement, and the chest movement direction and the abdomen movement direction are input into the learned model and the learned model outputs the determination result of synchronization between the chest and the abdomen when the input data is obtained. In the learning stage, the determination result of synchronization which is a correct label (ground-truth label) may be set by an instructor or the like. As described above, by using the learned model, it is possible to more accurately determine whether or not the movement of the chest 92 and the movement of the abdomen 94 are synchronized with each other.

For example, the result determination unit 140 determines whether or not the balance between the chest displacement and the abdomen displacement is good. The result determination unit 140 generates evaluation information indicating whether or not the balance between the chest displacement and the abdomen displacement is good. The result determination unit 140 determines whether or not the balance between the chest displacement and the abdomen displacement is good by using the ratio of the chest displacement to the abdomen displacement.

Specifically, when the abdomen displacement is set to x1 and the chest displacement is set to x2, a ratio of them, s, is expressed by the following Expression (1). That is, s is a ratio of the chest displacement x2 to the abdomen displacement x1.

$$s = x 2 / x 1 \qquad ( 1 )$$

Here, when s<0, that is, the sign of x1 does not coincide with the sign of x2, the result determination unit 140 determines that the balance between the chest displacement and the abdomen displacement is not good. In addition, when s>0, that is, the sign of x1 coincides with the sign of x2, the result determination unit 140 determines that the balance between the chest displacement and the abdomen displacement is not good if s<Th2. On the other hand, the result determination unit 140 determines that the balance between the chest displacement and the abdomen displacement is good if s≥Th2. Note that Th2 is a predetermined constant value larger than 0, such as 0.8. Th2 is adjusted according to the degree of mastery of the breathing method for each test subject 90.

For example, the result determination unit 140 determines whether or not the internal rotation of the rib of the test subject 90 is sufficiently performed during expiration. The result determination unit 140 generates evaluation information indicating whether or not the internal rotation of the rib of the test subject 90 is sufficiently performed during expiration. Specifically, the result determination unit 140 determines whether or not the chest width increase/decrease is below the threshold Th1 during expiration. When the chest width increase/decrease is below the threshold Th1 during expiration, the result determination unit 140 determines that the internal rotation of the rib is sufficiently performed. On the other hand, when the chest width increase/decrease is not below the threshold Th1 during expiration, the result determination unit 140 determines that the internal rotation of the rib is not sufficiently performed.

For example, the result determination unit 140 may evaluate the evaluation of the training result described above (for example, whether the chest and abdomen are synchronized) by comparing it with the training result in the past. That is, the result determination unit 140 evaluates the result of the current training of the test subject 90 by comparing it with the result of the past training of the test subject 90. The result determination unit 140 generates evaluation information related to the comparison between the result of the past training of the test subject 90 and the result of the current training of the test subject 90. That is, the result determination unit 140 generates evaluation information indicating whether or not the result of the current training of the subject is improved, compared with the result of the training of the test subject in the past.

For example, suppose that in the past training, breathing has been performed ten times, and synchronization between the chest and the abdomen has continued three times. In this case, the result determination unit 140 may determine that the breathing method of the test subject 90 is improved when synchronization between the chest and the abdomen continues three times or more in the current training. The result determination unit 140 may similarly evaluate, for the balance between the abdomen displacement and the chest displacement and for the internal rotation of the rib, the result of the current training of the test subject 90, compared with the result of the past training of the test subject 90.

The output control unit 150 performs control so that evaluation information related to the evaluation of the training result is output (Step S142). The output control unit 150 generates data indicating an instruction for outputting evaluation information (evaluation information output instruction data) in the manner described later, and transmits the data to the display device 40. Accordingly, the output control unit 150 performs control so that the evaluation information is output. The display device 40 outputs the evaluation information in accordance with the evaluation information output instruction data.

For example, the output control unit 150 may perform control so that the display device 40 displays a message (character string) or an image corresponding to the evaluation information. The output control unit 150 may perform control so that the display device 40 outputs a voice corresponding to the evaluation information. The output control unit 150 may perform control so that the display device 40 generates a vibration corresponding to the evaluation information. The output control unit 150 may perform control so that the display screen 200 turns to a color corresponding to the evaluation information.

The output control unit 150 may perform control so that the evaluation information is output in real time during breathing training. That is, the output control unit 150 may perform control so that the evaluation information is output at a timing when the training result that can change with the progress of breathing training is obtained. In this case, the output control unit 150 may perform control for displaying the evaluation information corresponding to the movement image, together with the movement image displayed on the display screen 200. Alternatively, the output control unit 150 may perform control for outputting the evaluation information (e.g., improvement method) that summarizes the training results after the training is completed.

The output control unit 150 may perform control so that the evaluation information indicating whether or not the movement of the chest 92 and the movement of the abdomen 94 are synchronized with each other is output. In this case, when the movement of the chest 92 and the movement of the abdomen 94 are synchronized with each other, the output control unit 150 may perform control so that the frame or background of the display screen 200 is displayed in green. On the other hand, when the movement of the chest 92 and the movement of the abdomen 94 are not synchronized with each other, the output control unit 150 may perform control so that the frame or background of the display screen 200 is displayed in red. For example, when the movement of the chest 92 and the movement of the abdomen 94 are synchronized with each other, the output control unit 150 may perform control so that a message such as “The chest and the abdomen are synchronized with each other” is output on the display screen 200 in character string or voice. On the other hand, when the movement of the chest 92 and the movement of the abdomen 94 are not synchronized with each other, the output control unit 150 may perform control so that a message such as “The chest and the abdomen are not synchronized with each other” is output on the display screen 200 in character string or voice. At this time, the output control unit 150 may perform control so that an alarm sound is output. For example, when the movement of the chest 92 and the movement of the abdomen 94 are synchronized with each other, the output control unit 150 may perform control so that the display device 40 generates vibration corresponding to the synchronization of the movement of the chest 92 and the movement of the abdomen 94. The output control unit 150 may perform control so that evaluation information is output in the same manner for the balance between the displacement of the abdomen and the displacement of the chest, and for the internal rotation of the rib.

The output control unit 150 may perform control so that a message for inducing breathing training is output. For example, when the training result is good (for example, the chest and abdomen are synchronized with each other), the output control unit 150 may perform control so that a message such as “That's the way” is output in character string or voice. On the other hand, when the training result is not good (for example, the chest and abdomen are not synchronized with each other), the output control unit 150 may perform control so that a message such as “Let's try harder” is output in character string or voice. When the internal-rotation of the rib is not sufficiently performed, the output control unit 150 may perform control so that a message such as “Please exhale fully” is output in character string or voice. In addition, when the result of the current training of the test subject is improved compared with the result of the past training of the test subject, the output control unit 150 may perform control so that a message such as “That's the way” is output in character string or voice.

When the training result is not good (for example, the chest and abdomen are not synchronized with each other), the output control unit 150 may perform control so that an alarm is output by image, voice, or the like. The output control unit 150 may perform control so that the number of alarms in one training session and the transition of the number of alarms are displayed. In addition, the output control unit 150 may perform control so that items of alarms are output. Note that the “items of alarms” indicate the contents (e.g., synchronization between the chest and the abdomen) in which alarms are output. The output control unit 150 may perform control so that the target value and the guidance of the breathing method are output.

In this way, the evaluation information is output under the control of the output control unit 150, and thereby it is possible to induce the motivation for breathing training of the test subject 90. In addition, the evaluation information related to the comparison between the results of the past training of the test subject 90 and the results of the current training of the test subject 90 is output under the control of the output control unit 150, and thereby the test subject 90 can easily recognize whether or not his/her own breathing method is improved. Therefore, the motivation of the test subject 90 to the breathing training can be further induced.

The information processing apparatus 100 determines whether or not to end the training (Step S150). Specifically, the information processing apparatus 100 determines whether or not an operation for ending the training is performed by a user (e.g., the instructor, etc.). For example, when the “measurement stop” button is operated in the setting screen 310 in the instructor-aimed display screen 300 shown in FIG. 16, the information processing apparatus 100 determines that the training is to be ended. On the other hand, when the “measurement stop” button is not operated in the setting screen 310 in the instructor-aimed display screen 300 shown in FIG. 16, the information processing apparatus 100 determines that the training is to be continued. If it is determined that training is to be continued (NO in S150), the processing flow returns to S106, and the processing in S106 to S142 is repeated. On the other hand, if it is determined that training is to be ended (YES in S150), the processing flow ends.

### Modified Example

Note that the present invention is not limited to the above-described example embodiments, and they may be modified as appropriate without departing from the scope and spirit of the invention. For example, one or more of the steps in the above-described flowchart may be omitted. Further, the order of one or more steps in the above-described flowchart may be changed as appropriate. For example, in the flowchart shown in FIG. 5, the processes in S120 and S122 may be omitted. Further, the processes in S130 and S132 may be performed before the processes in S110 and S112. Further, the processes in S112, S122 and S132 may be collectively performed after all the processes in S110, S120 and S130 have been completed. Further, a part of the processing in S140 and S142 may be performed after the training is completed.

Further, although the chest displacement image and the abdomen displacement image are displayed on the display device 40 in real time during the training in the above-described example embodiment, the present invention is not limited to this configuration. The chest displacement image and the abdomen displacement image may be displayed after the end of the training. In this case, the display device 40 may display a moving image of changes in the chest displacement image and the abdomen displacement image according to the time transition of the training.

The display system 20 may include a plurality of the photographing devices 30. In this case, the test subject 90 is photographed using the plurality of photographing devices 30. Accordingly, the test subject 90 can be photographed from a plurality of viewpoints, and thereby the occurrence of blind spots of the test subject 90 can be suppressed during photographing. Therefore, the displacement and the like can be detected more accurately.

Further, the display system 20 may be implemented by an apparatus in which two or more of the photographing device 30, the display device 40 and the information processing apparatus 100 are physically integrally configured. For example, the test subject 90 may perform breathing training by using one apparatus (such as a smartphone) including the photographing device 30, the display device 40 and the information processing apparatus 100. Accordingly, it is possible to perform breathing training without special equipment. For example, the test subject 90 can perform breathing training casually at home or the like.

The skeleton data may not be acquired by a device such as a smartphone. In this case, the test subject 90 may perform an operation to designate the chest region and the abdomen region in his/her photographed image 52. When one device including the photographing device 30, the display device 40 and the information processing apparatus 100 is used, breathing training may be performed by using a device including the photographing device 30 that cannot acquire three-dimensional data. For example, by photographing the test subject 90 by placing the device in the lateral direction of the test subject 90, the displacement and the like of the chest 92 and abdomen 94 of the test subject 90 can be detected. The apparatus implementing the display system 20 may only detect the displacement and the like during the breathing training, and display the displacement image and the like after the training is completed. The apparatus may output evaluation information by voice during the breathing training.

Further, although the display form indicating the movement image (the displacement image, the direction image, and the width increase/decrease image) is a red or blue color representation in the above-described example embodiment, the present invention is not limited to this configuration. The color representation can be arbitrarily set by a user. The display form is not limited to the color representation. For example, the display form may be grayscale (shades of black) or a plurality of kinds of patterns (hatching) or the like. That is, the display form may be such that the chest movement image and the abdomen movement image are visually the same when the chest movement and the abdomen movement are synchronized with each other. In other words, the display form may be such that the chest movement and the abdomen movement can be visually distinguished.

The program described above includes a set of instructions (or software codes) that, when read into a computer, causes the computer to perform one or more of the functions described in the example embodiments. The program may be stored in a non-transitory computer readable medium or in a physical storage medium. By way of example rather than limitation, a computer readable medium or a physical storage medium may include a random-access memory (RAM), a read-only memory (ROM), a flash memory, a solid-state drive (SSD), or other memory technology, a CD-ROM, a digital versatile disk (DVD), a Blu-ray (registered trademark) disc or other optical disc storages, a magnetic cassette, magnetic tape, and a magnetic disc storage or other magnetic storage devices. The program may be transmitted on a transitory computer readable medium or a communication medium. By way of example rather than limitation, the transitory computer readable medium or the communication medium may include electrical, optical, acoustic, or other forms of propagating signals.

The whole or part of the example embodiments disclosed above can be described as, but not limited to, the following supplementary notes.

### (Supplementary Note 1)

An information processing apparatus comprising:
acquisition means for acquiring image data indicating at least a chest and an abdomen of a test subject performing training of breathing;
detection means for detecting displacement of each of the chest and abdomen of the test subject by using the image data; and
display control means for performing control so that movement image indicating movement of each of the chest and abdomen in anteroposterior direction of the test subject is displayed based on the detected displacement of the chest and abdomen,
wherein the display control means performs control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

### (Supplementary Note 2)

The information processing apparatus according to Supplementary note 1, wherein
the display control means performs control so that a chest displacement image indicating the displacement in the anteroposterior direction of the chest is displayed as the chest movement image, and an abdomen displacement image indicating the displacement in the anteroposterior direction of the abdomen is displayed as the abdomen movement image, and
the display control means performs control so that the display form of the chest displacement image and the display form of the abdomen displacement image are same as each other when a sign of the displacement of the chest and a sign of the displacement of the abdomen are same as each other, and so that the display form of the chest displacement image and the display form of the abdomen displacement image are different from each other when the sign of the displacement of the chest and the sign of the displacement of the abdomen are different from each other.

### (Supplementary Note 3)

The information processing apparatus according to Supplementary note 1 or 2, wherein the display control means performs control so that the movement image that changes in accordance with progress of the training of breathing of the test subject is displayed during the progress of the training.

### (Supplementary Note 4)

The information processing apparatus according to any one of Supplementary notes 1 to 3, wherein the display control means performs control so that displacement in lateral direction of the chest of the test subject is displayed.

### (Supplementary Note 5)

The information processing apparatus according to any one of Supplementary notes 1 to 4, further comprising output control means for performing control so that evaluation information related to an evaluation of a result of the training is output.

### (Supplementary Note 6)

The information processing apparatus according to Supplementary note 5, wherein the output control means performs control so that the evaluation information related to a comparison between a result of the past training of the test subject and a result of the current training of the test subject is output.

### (Supplementary Note 7)

The information processing apparatus according to Supplementary note 5 or 6, wherein the output control means performs control so that the evaluation information indicating whether or not the movement of the chest and the movement of the abdomen are synchronized with each other is output.

### (Supplementary Note 8)

The information processing apparatus according to any one of Supplementary notes 5 to 7, wherein the output control means performs control so that the evaluation information indicating that balance between the displacement of the chest and the displacement of the abdomen is good is output when a ratio of the displacement in the anteroposterior direction of the chest to the displacement in the anteroposterior direction of the abdomen is larger than or equal to a predetermined threshold.

### (Supplementary Note 9)

The information processing apparatus according to any one of Supplementary notes 1 to 8, wherein
the display control means performs control so that a chest direction image indicating a direction of change of the displacement of the chest is displayed as the chest movement image and an abdomen direction image indicating a direction of change of the displacement of abdomen is displayed as the abdomen movement image, and
the display control means performs control so that the display form of the chest direction image and the display form of the abdomen direction image are same as each other when the direction of change of the displacement of the chest and the direction of change of the displacement of the abdomen are same as each other, and so that the display form of the chest direction image and the display form of the abdomen direction image are different from each other when the direction of change of the displacement of the chest and the direction of change of the displacement of the abdomen are different from each other.

### (Supplementary Note 10)

The information processing apparatus according to any one of Supplementary notes 1 to 9, wherein the display control means performs control so that a color representation of the chest movement image and a color representation of the abdomen movement image are same as each other when the movement of the chest and the movement of the abdomen are synchronized with each other, and so that the color representation of the chest movement image and the color representation of the abdomen movement image are different from each other when the movement of the chest and the movement of the abdomen are not synchronized with each other.

### (Supplementary Note 11)

The information processing apparatus according to any one of Supplementary notes 1 to 10, further comprising position specifying means for specifying a chest reference position which is a reference position of the chest in the anteroposterior direction and an abdomen reference position which is a reference position of the abdomen in the anteroposterior direction, wherein
the display control means performs control so that the display form of the chest movement image and the display form of the abdomen movement image are same as each other when the movement of the chest relative to the chest reference position and the movement of the abdomen relative to the abdomen reference position are synchronized with each other, and so that the display form of the chest movement image and the display form of the abdomen movement image are different from each other when the movement of the chest relative to the chest reference position and the movement of the abdomen relative to the abdomen reference position are not synchronized with each other.

### (Supplementary Note 12)

A display system comprising:
the information processing apparatus according to any one of Supplementary notes 1 to 11,
at least one photographing device photographing the test subject; and
a display device displaying the movement image, wherein
the acquisition means the image data from the photographing device, and
the display control means controls the display device.

### (Supplementary Note 13)

A display control method comprising:
acquiring image data indicating at least a chest and an abdomen of a test subject performing training of breathing;
detecting displacement of each of the chest and abdomen of the test subject by using the image data;
performing control so that movement image indicating movement of each of the chest and abdomen in anteroposterior direction of the test subject is displayed based on the detected displacement of the chest and abdomen; and
performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

### (Supplementary Note 14)

The display control method according to Supplementary note 13, further comprising:
performing control so that a chest displacement image indicating the displacement in the anteroposterior direction of the chest is displayed as the chest movement image, and an abdomen displacement image indicating the displacement in the anteroposterior direction of the abdomen is displayed as the abdomen movement image; and
performing control so that the display form of the chest displacement image and the display form of the abdomen displacement image are same as each other when a sign of the displacement of the chest and a sign of the displacement of the abdomen are same as each other, and so that the display form of the chest displacement image and the display form of the abdomen displacement image are different from each other when the sign of the displacement of the chest and the sign of the displacement of the abdomen are different from each other.

### (Supplementary Note 15)

The display control method according to Supplementary note 13 or 14, further comprising performing control so that the movement image that changes in accordance with progress of the training of breathing of the test subject is displayed during the progress of the training.

### (Supplementary Note 16)

The display control method according to any one of Supplementary notes 13 to 15, further comprising performing control so that displacement in lateral direction of the chest of the test subject is displayed.

### (Supplementary Note 17)

The display control method according to any one of Supplementary notes 13 to 16, further comprising performing control so that evaluation information related to an evaluation of a result of the training is output.

### (Supplementary Note 18)

The display control method according to Supplementary note 17, further comprising performing control so that the evaluation information related to a comparison between a result of the past training of the test subject and a result of the current training of the test subject is output.

### (Supplementary Note 19)

The display control method according to Supplementary note 17 or 18, further comprising performing control so that the evaluation information indicating whether or not the movement of the chest and the movement of the abdomen are synchronized with each other is output.

### (Supplementary Note 20)

The display control method according to any one of Supplementary notes 17 to 19, further comprising performing control so that the evaluation information indicating that balance between the displacement of the chest and the displacement of the abdomen is good is output when a ratio of the displacement in the anteroposterior direction of the chest to the displacement in the anteroposterior direction of the abdomen is larger than or equal to a predetermined threshold.

### (Supplementary Note 21)

The display control method according to any one of Supplementary notes 13 to 20, further comprising:
performing control so that a chest direction image indicating a direction of change of the displacement of the chest is displayed as the chest movement image and an abdomen direction image indicating a direction of change of the displacement of abdomen is displayed as the abdomen movement image, and
performing control so that the display form of the chest direction image and the display form of the abdomen direction image are same as each other when the direction of change of the displacement of the chest and the direction of change of the displacement of the abdomen are same as each other, and so that the display form of the chest direction image and the display form of the abdomen direction image are different from each other when the direction of change of the displacement of the chest and the direction of change of the displacement of the abdomen are different from each other.

### (Supplementary Note 22)

The display control method according to any one of Supplementary notes 13 to 21, further comprising performing control so that a color representation of the chest movement image and a color representation of the abdomen movement image are same as each other when the movement of the chest and the movement of the abdomen are synchronized with each other, and so that the color representation of the chest movement image and the color representation of the abdomen movement image are different from each other when the movement of the chest and the movement of the abdomen are not synchronized with each other.

### (Supplementary Note 23)

The display control method according to any one of Supplementary notes 13 to 22, further comprising:
specifying a chest reference position which is a reference position of the chest in the anteroposterior direction and an abdomen reference position which is a reference position of the abdomen in the anteroposterior direction; and
performing control so that the display form of the chest movement image and the display form of the abdomen movement image are same as each other when the movement of the chest relative to the chest reference position and the movement of the abdomen relative to the abdomen reference position are synchronized with each other, and so that the display form of the chest movement image and the display form of the abdomen movement image are different from each other when the movement of the chest relative to the chest reference position and the movement of the abdomen relative to the abdomen reference position are not synchronized with each other.

### (Supplementary Note 24)

A program for causing a computer to perform:
a step of acquiring image data indicating at least a chest and an abdomen of a test subject performing training of breathing;
a step of detecting displacement of each of the chest and abdomen of the test subject by using the image data;
a step of performing control so that movement image indicating movement of each of the chest and abdomen in anteroposterior direction of the test subject is displayed based on the detected displacement of the chest and abdomen, and performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

Although the present disclosure has been described with reference to the example embodiment, the present disclosure is not limited to the above. The configuration and details of the present disclosure can be modified in various ways that will be understood by those skilled in the art within the scope of the disclosure.

This application claims priority based on Japanese Patent Application 2021-136990 filed on Aug. 25, 2021 and all of its disclosures are incorporated herein.

### REFERENCE SIGNS LIST

1 INFORMATION PROCESSING APPARATUS
2 ACQUISITION UNIT
4 DETECTION UNIT
6 DISPLAY CONTROL UNIT
20 DISPLAY SYSTEM
30 PHOTOGRAPHING DEVICE
40 DISPLAY DEVICE
52 PHOTOGRAPHED IMAGE
90 TEST SUBJECT
92 CHEST
94 ABDOMEN
100 INFORMATION PROCESSING APPARATUS
112 IMAGE ACQUISITION UNIT
114 POSITION SPECIFYING UNIT
116 DISPLACEMENT DETECTION UNIT
118 MOVEMENT DIRECTION DETECTION UNIT
120 WIDTH DETECTION UNIT
130 DISPLAY CONTROL UNIT
140 RESULT DETERMINATION UNIT
150 OUTPUT CONTROL UNIT
200 DISPLAY SCREEN
210 CHEST DISPLACEMENT SCREEN
212 CHEST DISPLACEMENT IMAGE
220 ABDOMEN DISPLACEMENT SCREEN
222 ABDOMEN DISPLACEMENT IMAGE
230 TORSO DISPLACEMENT SCREEN
232bk TORSO REFERENCE POSITION DOT ROW
234 TORSO POSITION DOT ROW
240 DISPLACEMENT HISTORY SCREEN
250 CHEST DIRECTION SCREEN
252 CHEST DIRECTION IMAGE
260 ABDOMEN DIRECTION SCREEN
262 ABDOMEN DIRECTION IMAGE
270 CHEST WIDTH SCREEN
272 CHEST WIDTH IMAGE
280 ABDOMEN WIDTH SCREEN
282 ABDOMEN WIDTH IMAGE
300 INSTRUCTOR-AIMED DISPLAY SCREEN
310 SETTING SCREEN
322 RGB IMAGE DISPLAY SCREEN
324 DISTANCE IMAGE DISPLAY SCREEN
400 INSTRUCTOR-AIMED DISPLAY SCREEN
410 SETTING SCREEN
420 PHOTOGRAPHED IMAGE DISPLAY SCREEN

1. An information processing apparatus comprising:
hardware, including at least one processor and at least one memory;
acquisition unit implemented at least by the hardware and configured to acquire image data indicating at least a chest and an abdomen of a test subject performing training of breathing;
detection unit implemented at least by the hardware and configured to detect displacement of each of the chest and abdomen of the test subject by using the image data; and
display control unit implemented at least by the hardware and configured to perform control so that movement image indicating movement of each of the chest and abdomen in anteroposterior direction of the test subject is displayed based on the detected displacement of the chest and abdomen,
wherein the display control unit performs control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

2. The information processing apparatus according to claim 1, wherein
the display control unit performs control so that a chest displacement image indicating the displacement in the anteroposterior direction of the chest is displayed as the chest movement image, and an abdomen displacement image indicating the displacement in the anteroposterior direction of the abdomen is displayed as the abdomen movement image, and
the display control unit performs control so that the display form of the chest displacement image and the display form of the abdomen displacement image are same as each other when a sign of the displacement of the chest and a sign of the displacement of the abdomen are same as each other, and so that the display form of the chest displacement image and the display form of the abdomen displacement image are different from each other when the sign of the displacement of the chest and the sign of the displacement of the abdomen are different from each other.

3. The information processing apparatus according to claim 1, wherein the display control unit performs control so that the movement image that changes in accordance with progress of the training of breathing of the test subject is displayed during the progress of the training.

4. The information processing apparatus according to claim 1, wherein the display control unit performs control so that displacement in lateral direction of the chest of the test subject is displayed.

5. The information processing apparatus according to claim 1, further comprising output control unit implemented at least by the hardware and configured to perform control so that evaluation information related to an evaluation of a result of the training is output.

6. The information processing apparatus according to claim 5, wherein the output control unit performs control so that the evaluation information related to a comparison between a result of the past training of the test subject and a result of the current training of the test subject is output.

7. The information processing apparatus according to claim 5, wherein the output control unit performs control so that the evaluation information indicating whether or not the movement of the chest and the movement of the abdomen are synchronized with each other is output.

8. The information processing apparatus according to claim 5, wherein the output control unit performs control so that the evaluation information indicating that balance between the displacement of the chest and the displacement of the abdomen is good is output when a ratio of the displacement in the anteroposterior direction of the chest to the displacement in the anteroposterior direction of the abdomen is larger than or equal to a predetermined threshold.

9. The information processing apparatus according to claim 1, wherein
the display control unit performs control so that a chest direction image indicating a direction of change of the displacement of the chest is displayed as the chest movement image and an abdomen direction image indicating a direction of change of the displacement of abdomen is displayed as the abdomen movement image, and
the display control unit performs control so that the display form of the chest direction image and the display form of the abdomen direction image are same as each other when the direction of change of the displacement of the chest and the direction of change of the displacement of the abdomen are same as each other, and so that the display form of the chest direction image and the display form of the abdomen direction image are different from each other when the direction of change of the displacement of the chest and the direction of change of the displacement of the abdomen are different from each other.

10. The information processing apparatus according to claim 1, wherein the display control unit performs control so that a color representation of the chest movement image and a color representation of the abdomen movement image are same as each other when the movement of the chest and the movement of the abdomen are synchronized with each other, and so that the color representation of the chest movement image and the color representation of the abdomen movement image are different from each other when the movement of the chest and the movement of the abdomen are not synchronized with each other.

11. The information processing apparatus according to claim 1, further comprising position specifying unit implemented at least by the hardware and configured to specify a chest reference position which is a reference position of the chest in the anteroposterior direction and an abdomen reference position which is a reference position of the abdomen in the anteroposterior direction, wherein
the display control unit performs control so that the display form of the chest movement image and the display form of the abdomen movement image are same as each other when the movement of the chest relative to the chest reference position and the movement of the abdomen relative to the abdomen reference position are synchronized with each other, and so that the display form of the chest movement image and the display form of the abdomen movement image are different from each other when the movement of the chest relative to the chest reference position and the movement of the abdomen relative to the abdomen reference position are not synchronized with each other.

12. (canceled)

13. A display control method comprising:
acquiring image data indicating at least a chest and an abdomen of a test subject performing training of breathing;
detecting displacement of each of the chest and abdomen of the test subject by using the image data;
performing control so that movement image indicating movement of each of the chest and abdomen in anteroposterior direction of the test subject is displayed based on the detected displacement of the chest and abdomen; and
performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.

14. The display control method according to claim 13, further comprising:
performing control so that a chest displacement image indicating the displacement in the anteroposterior direction of the chest is displayed as the chest movement image, and an abdomen displacement image indicating the displacement in the anteroposterior direction of the abdomen is displayed as the abdomen movement image; and
performing control so that the display form of the chest displacement image and the display form of the abdomen displacement image are same as each other when a sign of the displacement of the chest and a sign of the displacement of the abdomen are same as each other, and so that the display form of the chest displacement image and the display form of the abdomen displacement image are different from each other when the sign of the displacement of the chest and the sign of the displacement of the abdomen are different from each other.

15. The display control method according to claim 13, further comprising performing control so that the movement image that changes in accordance with progress of the training of breathing of the test subject is displayed during the progress of the training.

16. The display control method according to claim 13, further comprising performing control so that displacement in lateral direction of the chest of the test subject is displayed.

17. The display control method according to claim 13, further comprising performing control so that evaluation information related to an evaluation of a result of the training is output.

18. The display control method according to claim 17, further comprising performing control so that the evaluation information related to a comparison between a result of the past training of the test subject and a result of the current training of the test subject is output.

19. The display control method according to claim 17, further comprising performing control so that the evaluation information indicating whether or not the movement of the chest and the movement of the abdomen are synchronized with each other is output.

20. The display control method according to claim 17, further comprising performing control so that the evaluation information indicating that balance between the displacement of the chest and the displacement of the abdomen is good is output when a ratio of the displacement in the anteroposterior direction of the chest to the displacement in the anteroposterior direction of the abdomen is larger than or equal to a predetermined threshold.

21. (canceled)

22. (canceled)

23. (canceled)

24. A non-transitory computer readable medium storing a program for causing a computer to perform:
a step of acquiring image data indicating at least a chest and an abdomen of a test subject performing training of breathing;
a step of detecting displacement of each of the chest and abdomen of the test subject by using the image data;
a step of performing control so that movement image indicating movement of each of the chest and abdomen in anteroposterior direction of the test subject is displayed based on the detected displacement of the chest and abdomen, and performing control so that a display form of a chest movement image which is the movement image of the chest and a display form of an abdomen movement image which is the movement image of the abdomen are same as each other when the movement of the chest and the movement of the abdomen are synchronized with each other, and so that the display form of the chest movement image and the display form of the abdomen movement image are different from each other when the movement of the chest and the movement of the abdomen are not synchronized with each other.
